(19) United States
(12) Patent Application Publication
(10) Pub. No.: US 2020/0030215 A1
(43) Pub. Date: Jan. 30, 2020

(54) AQUEOUS COMPOSITION COMPRISING A COMPOUND OF THIOLACTONE TYPE AND A SILICONE AND PROCESS FOR TREATING KERATIN MATERIALS WITH THE COMPOSITION

(71) Applicant: L'Oreal, Paris (FR)

(72) Inventors: Sébastien VOISIN, Saint-Ouen (FR); Nicolas DAUBRESSE, Saint-Ouen (FR); Manon CHAUMONTET, Sain-Ouen (FR)

(21) Appl. No.: 16/469,225

(22) PCT Filed: Dec. 21, 2017
(86) PCT No.: PCT/EP2017/084083

§ 371 Date: Jun. 13, 2019

(30) Foreign Application Priority Data
Dec. 21, 2016 (FR) 1662990

(51) Int. Cl. A61K 8/49; A61K 8/898; A61Q 5/06

(52) U.S. Cl. CPC A61K 8/4986; A61K 2800/30; A61Q 5/06; A61K 8/898

(57) ABSTRACT

The present invention relates to a composition comprising: (a) one more compound(s) of thiolactone type, and (b) one or more silicones.

The present invention relates to a composition comprising at least one compound of thiolactone type and at least one silicone and to a process for treating keratin materials, in particular human keratin fibres, comprising the application of the composition, in particular for shaping keratin fibres, and in particular for straightening/relaxing the hair.

In the hair field, consumers wish to have available compositions which make it possible to introduce a temporary change to their head of hair, while targeting good shape retention of the effect produced. In general, it is desired for the change to withstand shampooing for at least fifteen days or even more, depending on the nature of said change.

Heat treatments are generally used to modify the shape of the head of hair in a long-lasting manner. These treatments allow a visual modification of the appearance of the hairstyle, combining a decrease in the degree of frizziness, a reduction in overall volume of the head of hair, a decrease in little curls, a gain in manageability, a straighter overall appearance, a substantial gain in sheen, and a resistance to humidity and to heat in order to maintain the hairstyle throughout the day.

Moreover, this type of treatment has the advantage of facilitating the daily maintenance of the head of hair, with the use of fewer care products, in particular rinse-out care products such as hair conditioners or masks, or leave-in care products such as sera, care creams or balms, or taming mousses. Drying of the hair is facilitated, with a much shortened blow-drying time and a decrease in the daily use of flat irons, in terms both of time and intensity. This thus makes it possible to limit the risks of damaging the hair through combined factors of mechanical and thermal stress.

Several techniques are combined with these heat treatments. A first technique is based on the use of compositions based on thiol-based reducing agents. These techniques require strict adherence to the application conditions recommended by the suppliers, in particular in terms of amount and leave-in time. In addition, they may be contraindicated on hair that is too sensitized and may not be compatible with same-day application of other treatments, such as dyeing or bleaching operations. Moreover, they have an unpleasant smell.

Another technique is based on the use of compositions based on formol (or formaldehyde) and derivatives thereof. These treatments have the particular feature of being robust, perfectly compatible with all the other conventional hair treatments, such as the thiol-based straightening operations previously mentioned, alkaline relaxing operations, dyeing or bleaching operations of all types, performed before or after. They provide the hair with excellent manageability, a very bright sheen and easy daily care. However, in the event of repeated applications, further damage to the hair occurs, which can lead to breaking of the hairs. Furthermore, for toxicological reasons, the use of some of these compounds is now prohibited and/or regulated. It is therefore increasingly sought to avoid the use of such substances, which may prove to be aggressive to the hair and other keratin materials.

Another technique is based on the use of compositions based on acids, and most particularly on the use of glyoxylic acid. Patent application WO 2011/104 282 thus proposed a novel process for semi-permanently straightening the hair, which consists in applying an α-keto acid solution to the hair for 15 to 120 minutes, then drying and, finally, straightening the head of hair with an iron at a temperature of about 200° C. The α-keto acid employed is preferably glyoxylic acid. However, it has been noted that glyoxylic acid may not be well tolerated, in particular when the scalp is sensitive and/or irritated. Its volatility, amplified by the use of heat from the iron, can also be a problem. Moreover, the prior art compositions can modify the hair and/or modify the colour thereof, and most particularly can cause unwanted changes in artificial colourings such as oxidation colours.

Treatments using a composition comprising a base combined with a heat treatment have also been proposed for straightening the hair. Such treatments make it possible to obtain good relaxing of curls, but can lead to impairment of the hair fibre. EP 1837010 especially describes a straightening/relaxing process using a composition comprising sodium hydroxide and a heat treatment. WO 2007/144707 describes a straightening/relaxing process using a composition comprising a non-hydroxylated base such as monoethanolamine or ethylenediamine, combined with a heat treatment. WO 2009/117344 also describes a straightening/relaxing process using a composition comprising a non-hydroxylated base and a protein-denaturing agent, combined with a heat treatment. In order to limit hair fibre impairment, it has also been proposed to use compositions comprising weak acids at alkaline pH, combined with a heat treatment. WO 2010/049434 describes, for example, a straightening/relaxing process in which a composition comprising a dicarboxylic acid, such as maleic acid, and a heat treatment are applied.

There is still a need to develop a process for treating keratin materials, in particular keratin fibres such as the hair, more particularly a process for shaping the hair, in particular straightening the hair, in a way that is efficient and long-lasting, while at the same time limiting degradation of the hair, in particular in a way that is persistent with respect to successive shampooing operations.

The applicant has now discovered that the use of an aqueous composition comprising at least one compound of thiolactone type and at least one silicone makes it possible to achieve the desired long-lasting shaping properties.

Thus, a subject of the present invention is a composition comprising:

(a) one or more compound(s) chosen from the compounds of formula (I) below:

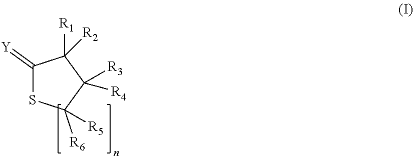

and also the organic or mineral acid or base salts thereof, the optical isomers thereof, the geometrical isomers thereof, the tautomers thereof, and the solvates thereof such as hydrates;
in which formula (I):
Y represents an oxygen or sulfur atom or a group NR with R representing a hydrogen atom or a (C₁-C₆)alkyl or hydroxy(C₁-C₆)alkyl group; preferably, Y represents an oxygen atom;
R₁, R₂, R₃, R₄, R₅ and R₆, which may be identical or different, represent:
i) a hydrogen atom,
ii) hydroxyl,
iii) amino —NR′R″, with R′ and R″, which may be identical or different, representing a hydrogen atom or a (C₁-C₆)alkyl or hydroxy(C₁-C₆)alkyl group;
iv) cyano,
v) —(Y′)ₚ—C(Y″)—(Y′″)_{q}—R₇ with R₇ representing a hydrogen atom or a (C₁-C₆)alkyl group, p and q, which may be identical or different, being equal to 0 or 1, preferably with p+q=0 or 1, Y′, Y″ and Y′″, which may be identical or different, representing an oxygen or sulfur atom, or NR with R as defined previously, preferably Y′, Y″ and Y′″ represent an oxygen atom;
vi) optionally substituted (C₁-C₆)alkyl,
vii) optionally substituted (C₂-C₆)alkenyl,
viii) optionally substituted (C₁-C₆)alkoxy, preferably, said alkyl, alkenyl or alkoxy groups being optionally substituted with one or more groups chosen from hydroxyl and —(O)ₜ—C(O)—R₈ with t being equal to 0 or 1, R₈ represents a) a hydrogen atom, b) a hydroxyl group, c) (C₁-C₆)alkyl, d) (C₁-C₆)alkoxy, e) (di)(C₁-C₆)(alkyl)amino, f) (di)hydroxy(C₁-C₆)alkylamino; or alternatively:
ix) R₁ and R₃ together form a covalent bond;
x) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl optionally substituted with one or more hydroxyl groups, and d) (C₂-C₆)alkenyl, e) —(Y′)ₚ—C(Y″)—(Y′″)_{q}—R₇ with Y′, Y″, Y′″, p, q and R₇ being as defined previously;
n represents an integer between 0 and 6 inclusive, particularly between 0 and 4 inclusive, more particularly between 0 and 3 inclusive, preferably n is 1 or 2, and more preferentially n is 1; it being understood that:
   when n is greater than or equal to 2, then the contiguous groups C(R₅)—R₆ may be identical or different; and
      (b) one or more silicones.

Another subject of the invention is a process for treating keratin materials, in particular keratin fibres, especially human keratin fibres such as the hair, for shaping keratin fibres, in particular straightening and/or relaxing said fibres, using the composition as defined previously.

The composition of the invention makes it possible in particular to obtain good long-lasting relaxation of curls, in particular with respect to shampooing operations, while limiting the degradation of the hair. It also makes it possible to limit the formation of frizz in the presence of moisture.

Other characteristics and advantages of the invention will emerge more clearly on reading the description and the examples that follow.

In the following text, unless indicated otherwise:
the term “organic or mineral acid salt” is intended to mean cosmetically acceptable organic or mineral acid salts, more particularly the salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
the term “alkyl” is intended to mean a linear or branched hydrocarbon-based radical containing from 1 to 8 carbon atoms, in particular from 1 to 6 carbon atoms, preferably from 1 to 4 carbon atoms, for example methyl, ethyl, n-propyl, isopropyl, butyl, n-pentyl, n-hexyl, preferably methyl;
the term “alkenyl” is intended to mean a linear or branched hydrocarbon-based radical containing from 2 to 8 carbon atoms, in particular from 2 to 6 carbon atoms, preferably from 2 to 4 carbon atoms, and comprising at least one or more conjugated or non-conjugated unsaturations, such as ethylenyl, n-propylenyl, isopropylenyl, butylenyl, n-pentylenyl, n-hexylenyl;
the term “alkoxy” is intended to mean an alkyl oxy group with “alkyl” or “alkenyl” as defined previously;
the term “optionally substituted” preceding alkyl, alkenyl or alkoxy groups is intended to mean that said groups may be substituted on the hydrocarbon-based radical with one or more groups, which may be identical or different, chosen from i) hydroxyl, ii) thiol, iii) halogen, iv) (C₁-C₄)alkoxy, v) hydroxy(C₂-C₄)alkoxy; vi) (di)hydroxy(C₁-C₄)(alkyl)amino, vii) cyano, viii) nitro(so), ix) Rₐ—Zₐ—C(Z_{b})—Z_{c}— and x) Rₐ—Zₐ—S(O)ₜ—Z_{c}— with Z_{b} representing an oxygen or sulfur atom or a group NRₐ′, Zₐ and Z_{c}, which may be identical or different, representing a bond, an oxygen or sulfur atom or a group NRₐ; Rₐ representing a hydrogen atom or a (C₁-C₄)alkyl group and R_{a′} representing a hydrogen atom or an alkyl group and t being 1 or 2;
moreover, the addition salts that may be used in the context of the invention are especially chosen from salts of addition with a cosmetically acceptable base such as the basifying agents as defined below, for instance alkali metal hydroxides such as sodium hydroxide or potassium hydroxide, aqueous ammonia, amines or alkanolamines;
the term “relaxing” includes, according to the invention, the relaxing, straightening or uncurling of Caucasian or African hair. The term “to relax” includes, according to the invention, the act of relaxing, straightening or uncurling of Caucasian or African hair;
the expression “at least one” is equivalent to “one or more”; and
the expression “inclusive” for a range of concentrations means that the limits of the range are included in the defined interval.

The composition according to the invention comprises one or more compound(s) of formula (I).

According to one particular embodiment of the invention, the compound(s) of formula (I) are such that n is 1 or 2, and preferably of formula (Ia):

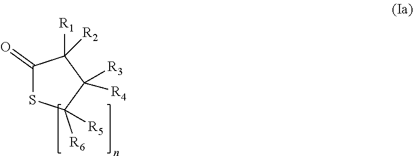

and also the organic or mineral acid or base salts thereof, the optical isomers thereof, the geometrical isomers thereof, the tautomers thereof, and the solvates thereof such as hydrates;
in which formula (Ia):
R₁, R₂, R₃, R₄, R₅ and R₆, which may be identical or different, represent:
i) a hydrogen atom,
ii) hydroxyl,
iii) cyano,
iv) amino —NR′R″, R′ and R″, which may be identical or different, representing a hydrogen atom or a (C₁-C₆)alkyl or hydroxy(C₁-C₆)alkyl group;
v) —(Y′)ₚ—C(O)—(Y″)_{q}—R₇ with R₇ representing a hydrogen atom or a (C₁-C₆)alkyl group, p and q, which may be identical or different, being equal to 0 or 1, with Y′ and Y″, which may be identical or different, representing an oxygen atom or NR with R as defined previously, preferably with p+q=0 or 1,
vi) (C₁-C₆)alkyl optionally substituted with one or more groups chosen from hydroxyl and —C(O)—R₈ with R₈ representing a) a hydrogen atom, b) a hydroxyl group, c) (C₁-C₆)alkoxy,
vii) (C₂-C₆)alkenyl,
viii) (C₁-C₆)alkoxy, or alternatively:
ix) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl.

According to a particular embodiment of the invention, the compound(s) of formula (I) are such that n is 1, and preferably of formula (I′a):

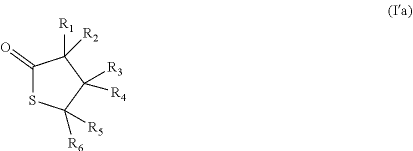

and also the organic or mineral acid or base salts thereof, the optical isomers thereof, the geometrical isomers thereof, the tautomers thereof, and the solvates thereof such as hydrates;
in which formula (I′a):
R₁, R₂, R₃, R₄, R₅ and R₆, which may be identical or different, are as defined previously, and in particular represent:
i) a hydrogen atom,
ii) hydroxyl,
iii) cyano,
iv) amino —NR′R″, R′ and R″, which may be identical or different, representing a hydrogen atom or a (C₁-C₆)alkyl or hydroxy(C₁-C₆)alkyl group;
v) —(Y′)ₚ—C(O)—(O)_{q}—R₇ with R₇ representing a hydrogen atom or a (C₁-C₆)alkyl group, p and q, which may be identical or different, being equal to 0 or 1, with Y′ representing an oxygen atom or NR with R as defined previously, preferably with p+q=0 or 1, vi) optionally substituted (C₁-C₆)alkyl,
vii) (C₂-C₆)alkenyl,
viii) (C₁-C₆)alkoxy,
   said alkyl groups being optionally substituted with one or more groups chosen from hydroxyl and —(O)ₜ—C(O)—R₈ with t being equal to 0 or 1, R₈ representing a) a hydrogen atom, b) a hydroxyl group, c) (C₁-C₆)alkyl, d) (C₁-C₆)alkoxy, e) (di)(C₁-C₆)(alkyl)amino, f) (di)hydroxy(C₁-C₆)alkylamino;
   or alternatively:
ix) R₁ and R₂, and/or R₃ and R₄, and/or R₅ and R₆ form, together with the carbon atom that bears them, a double bond oxo ═O or ═C(R₉)—R₁₀ with R₉ and R₁₀ representing i) a hydrogen atom or a group chosen from ii) hydroxyl, iii) (C₁-C₆)alkyl optionally substituted with one or more hydroxyl groups, in particular, R₃ and R₄ form, together with the oxygen atom, an oxo group and/or in particular, R₁ and R₂ represent, together with the carbon atom, ═C(R₉)—R₁₀.

According to one particular embodiment of the invention, the compound(s) of formula (I) are such that n is 1 or 2, and is of formula (Ib):

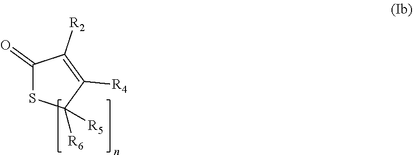

and also the organic or mineral acid or base salts thereof, the optical isomers thereof, the geometrical isomers thereof, the tautomers thereof, and the solvates thereof such as hydrates;
in which formula (Ib):
R₂, R₄, R₅ and R₆, which may be identical or different, represent:
i) a hydrogen atom,
ii) hydroxyl,
iii) cyano,
iv) amino —NR′R″, R′ and R″, which may be identical or different, representing a hydrogen atom or a (C₁-C₆)alkyl or hydroxy(C₁-C₆)alkyl group;
v) —(Y′)ₚ—C(O)—(O)_{q}—R₇ with R₇ representing a hydrogen atom or a (C₁-C₆)alkyl group, p and q, which may be identical or different, being equal to 0 or 1, with p+q=0 or 1, with Y′ representing an oxygen atom or NR with R as defined previously,
vi) (C₁-C₆)alkyl optionally substituted with one or more groups chosen from hydroxyl and —C(O)—R₈ with R₈ representing a) a hydrogen atom, b) a hydroxyl group, c) (C₁-C₆)alkoxy,
vii) (C₂-C₆)alkenyl,
viii) (C₁-C₆)alkoxy, or alternatively:
ix) R₅ and R₆ form, together with the carbon atom that bears them, a double bond oxo ═O or ═C(R₉)—R₁₀ with R₉ and R₁₀ representing a hydrogen atom or a (C₁-C₆)alkyl group.

According to one particular embodiment of the invention, the compound(s) of formula (I) are of formula (Ic):

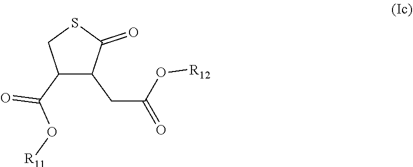

and also the organic or mineral acid or base salts thereof, the optical isomers thereof, the geometrical isomers thereof, the tautomers thereof, and the solvates thereof such as hydrates;
in which formula (Ic):
R₁₁ and R₁₂, which may be identical or different, represent a hydrogen atom or a (C₁-C₆)alkyl group, such as methyl or ethyl. More preferentially, R₁₁ and R₁₂ represent a hydrogen atom.

According to one particular embodiment, the compounds of formula (I′c) are chosen from:

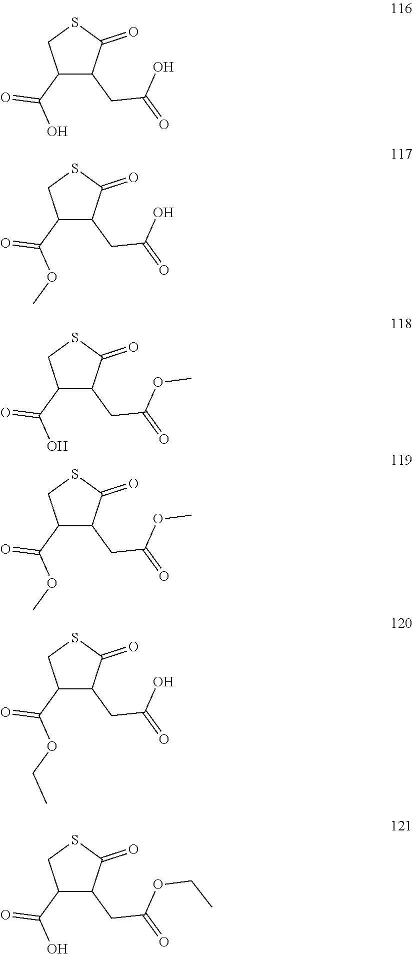

and also the organic or mineral base salts, the optical isomers, and the solvates such as hydrates.

According to a more particular embodiment of the invention, the compound(s) of formula (I) are of formula (I′a) or (Ib) and n is 1.

More preferentially, the compounds of formula (I) of the invention are chosen from the following compounds:

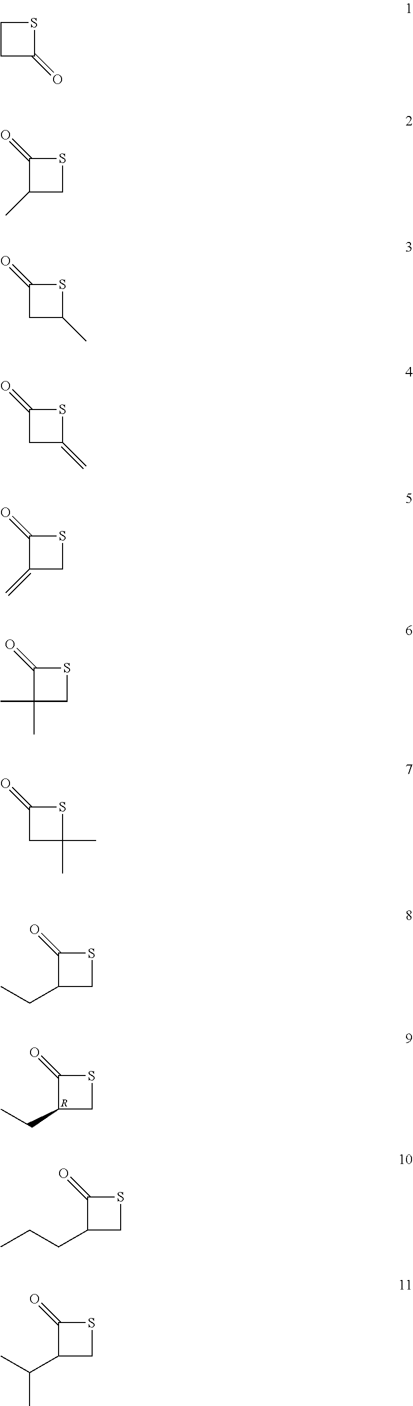

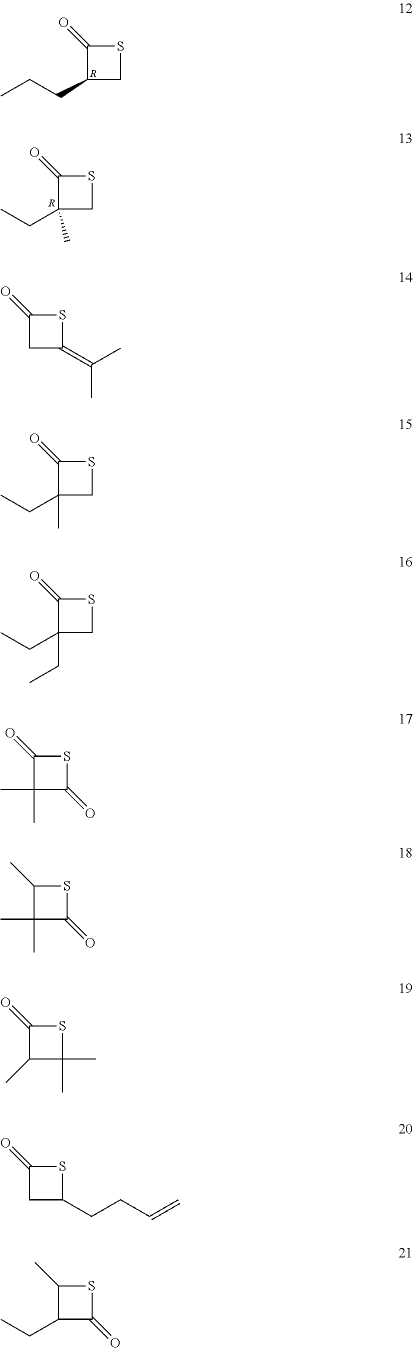

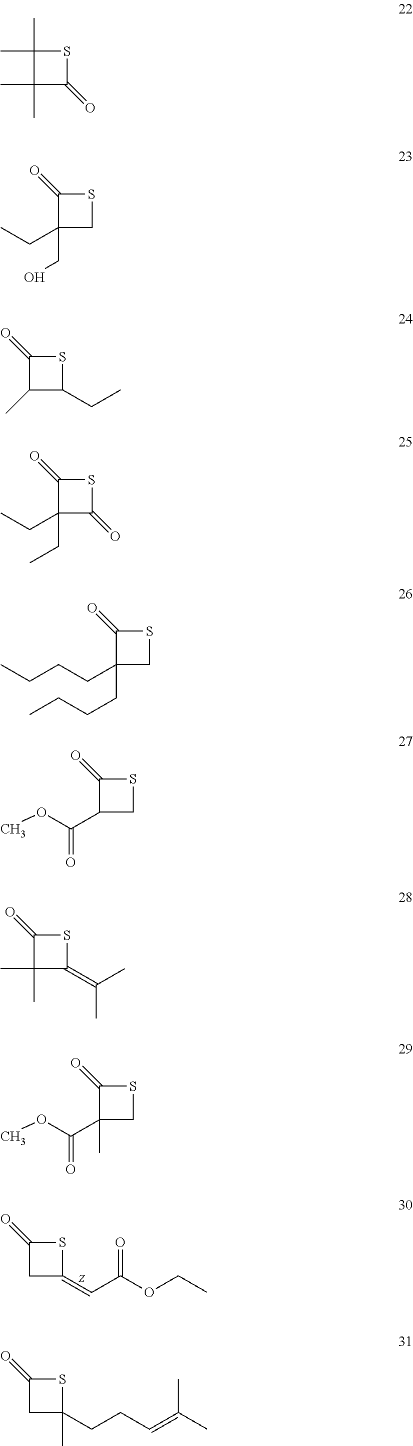

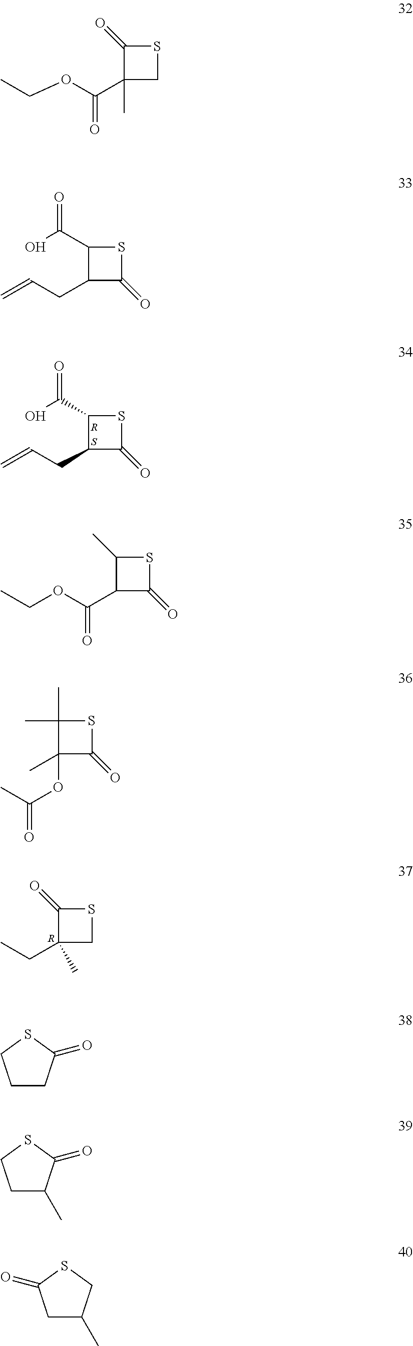

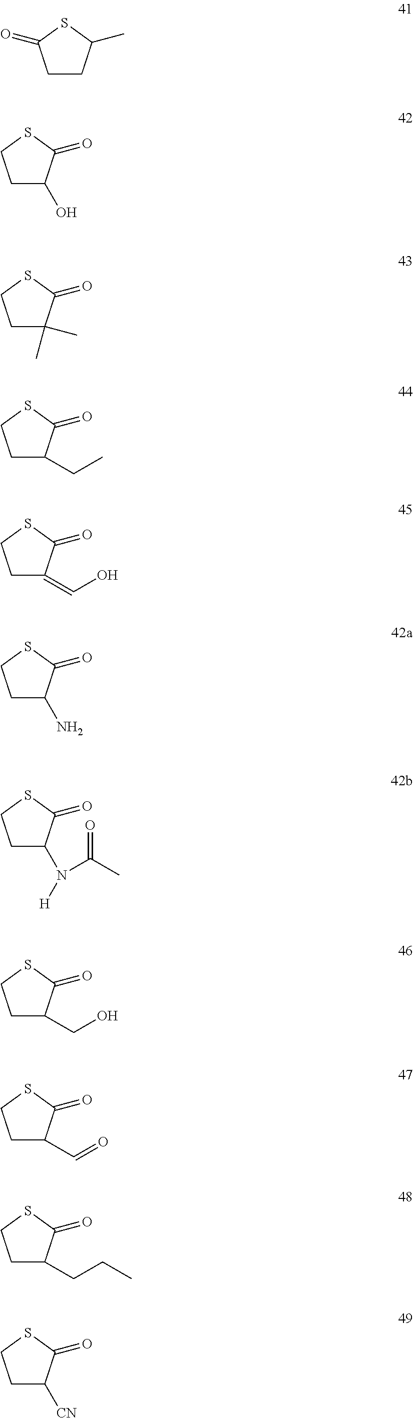

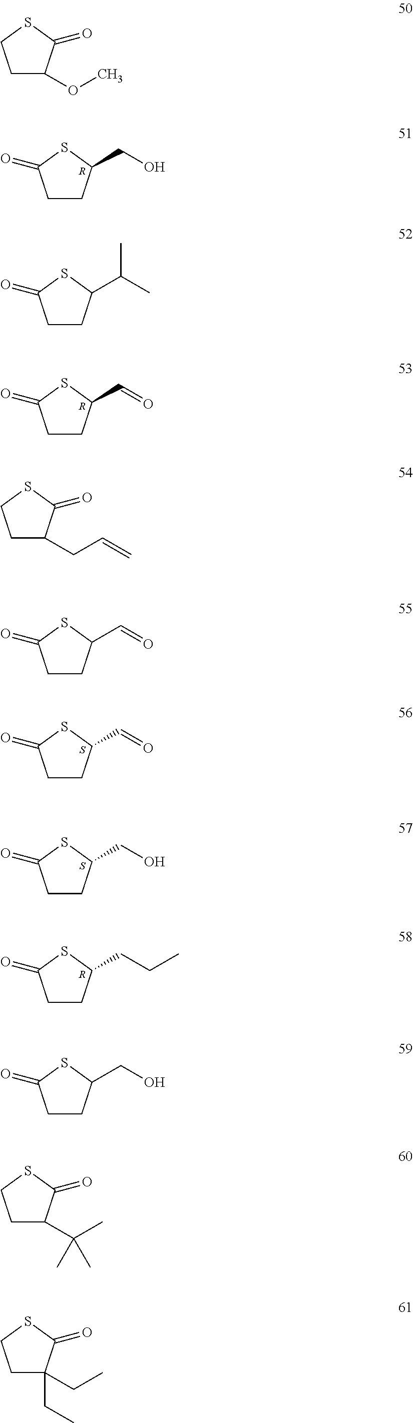

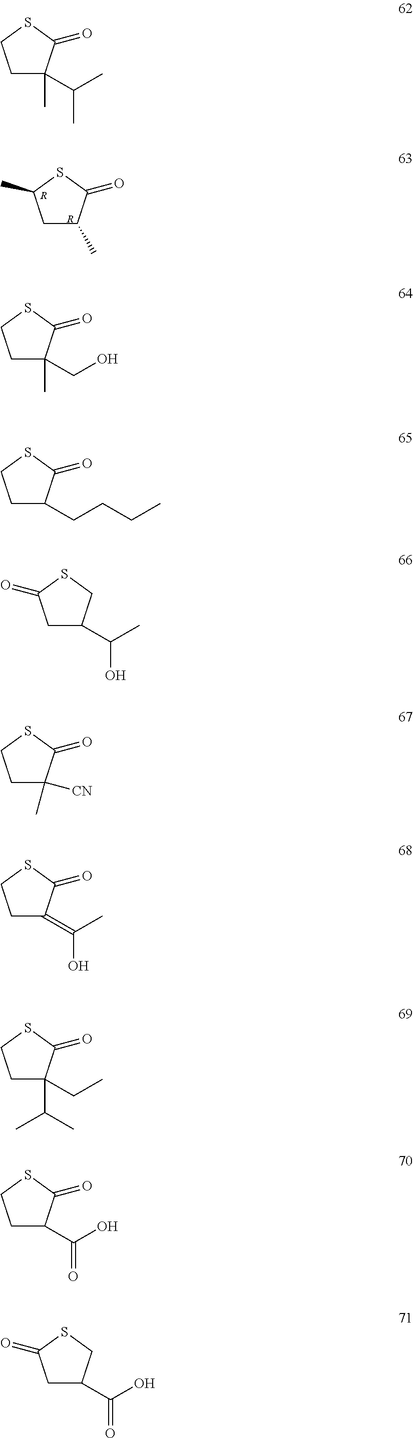

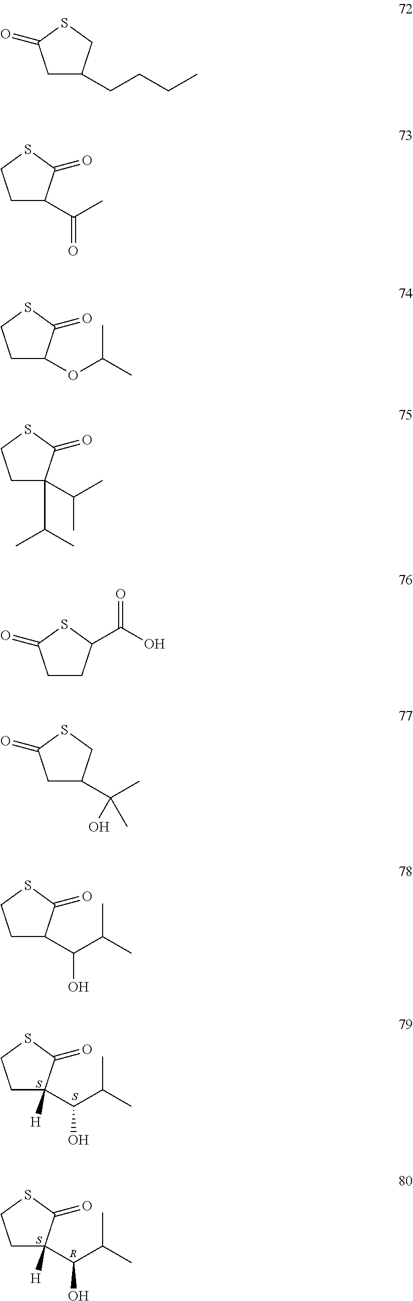

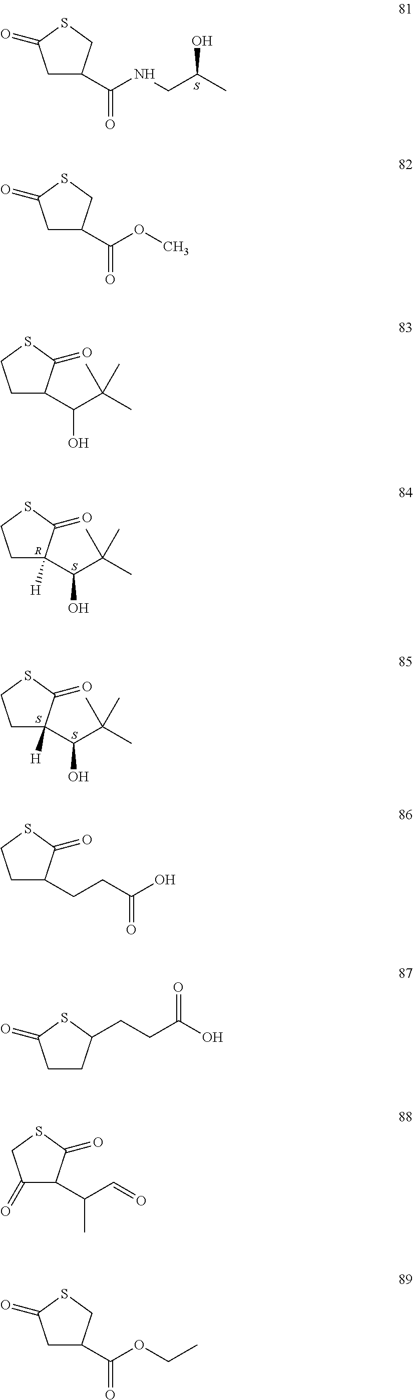

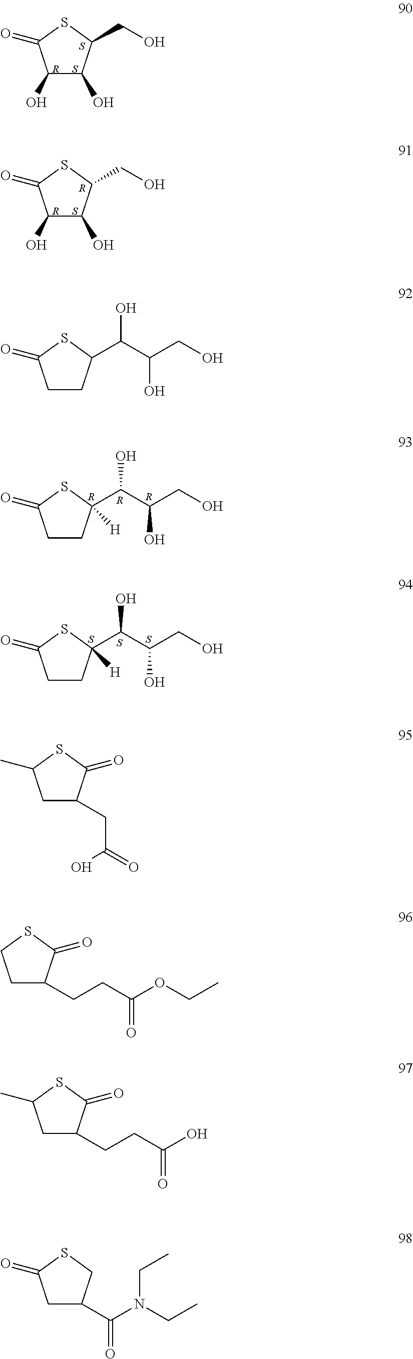

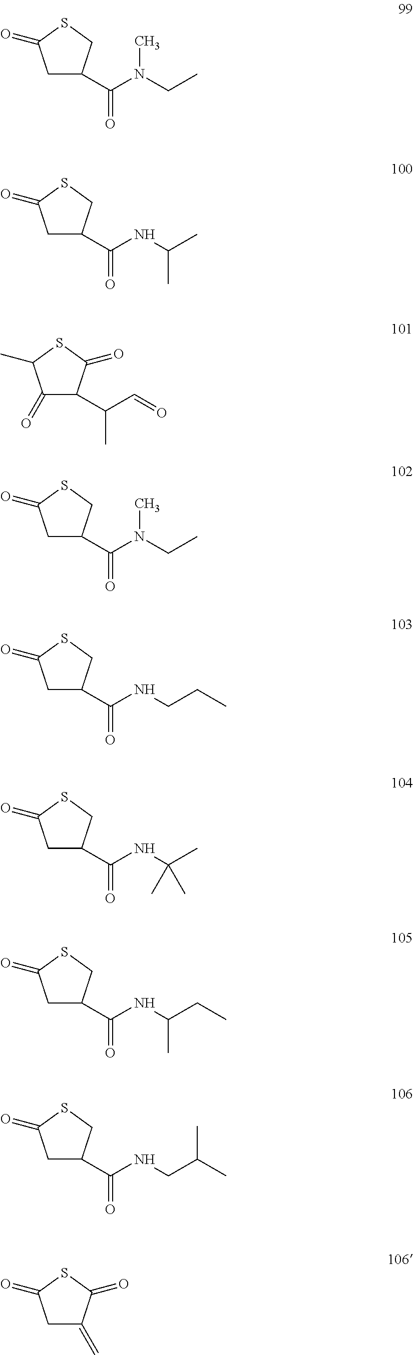

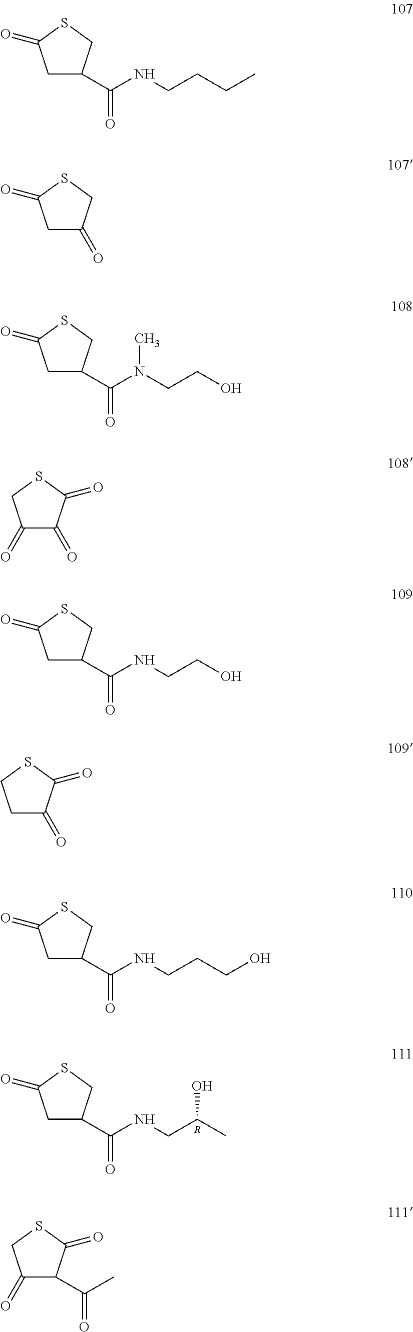

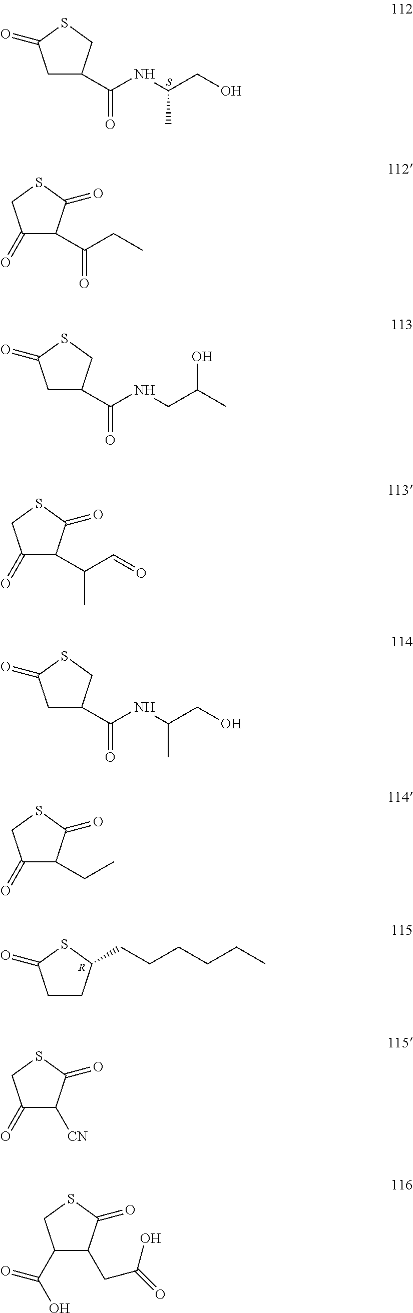

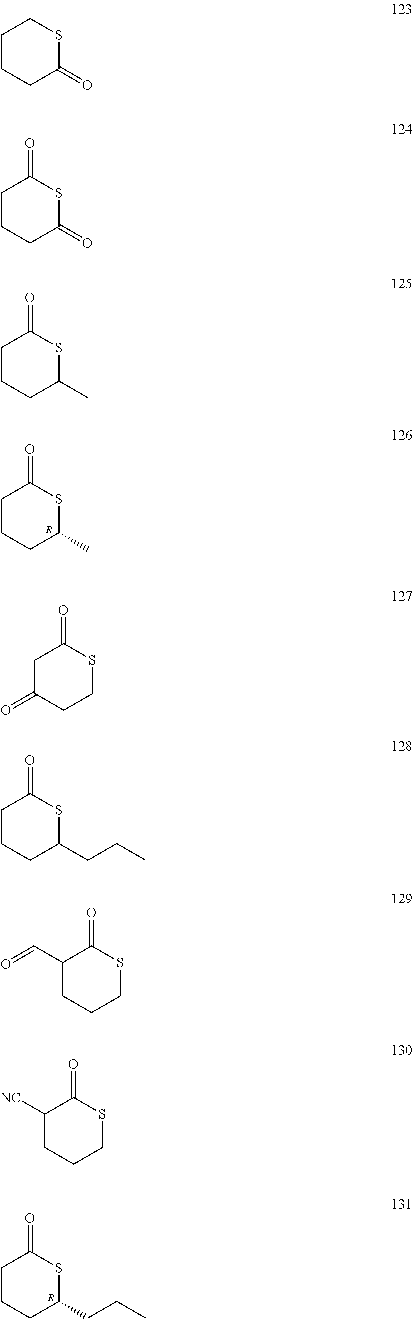

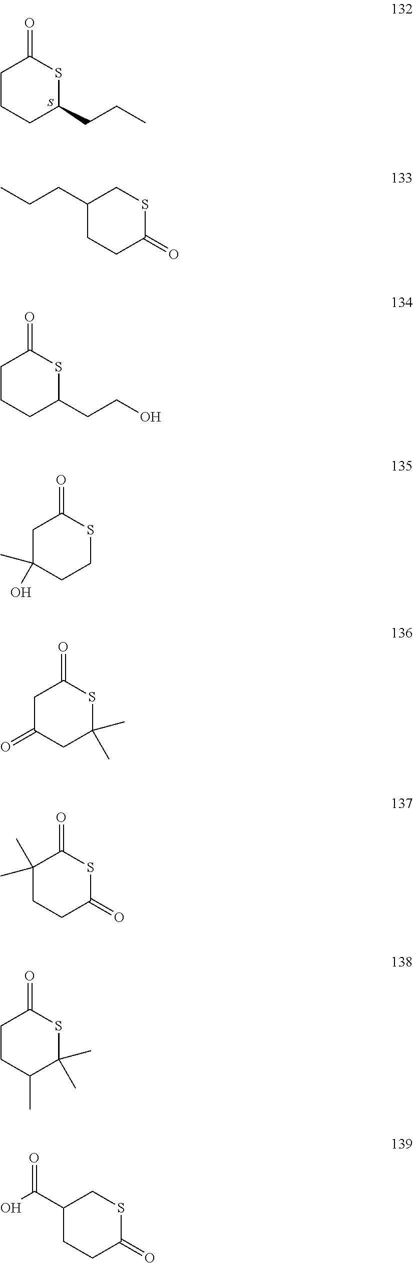

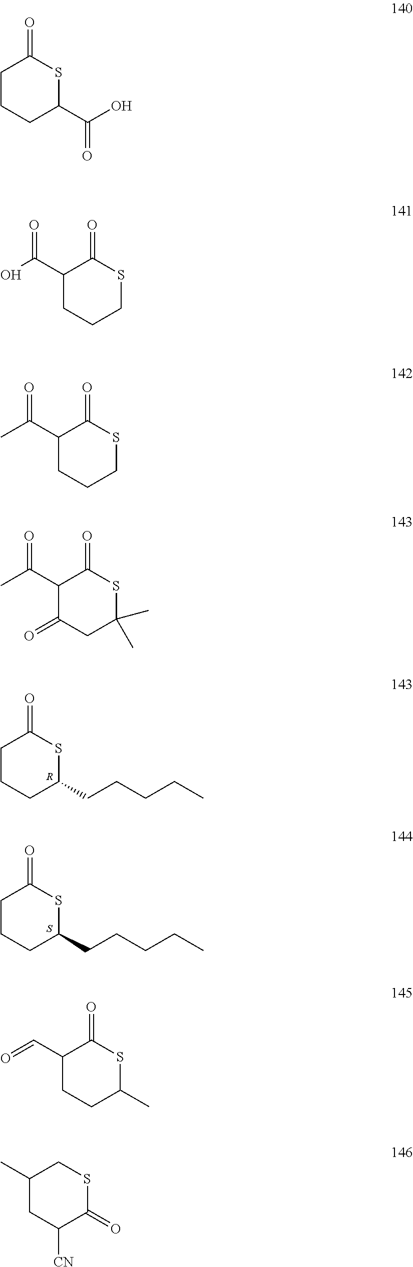

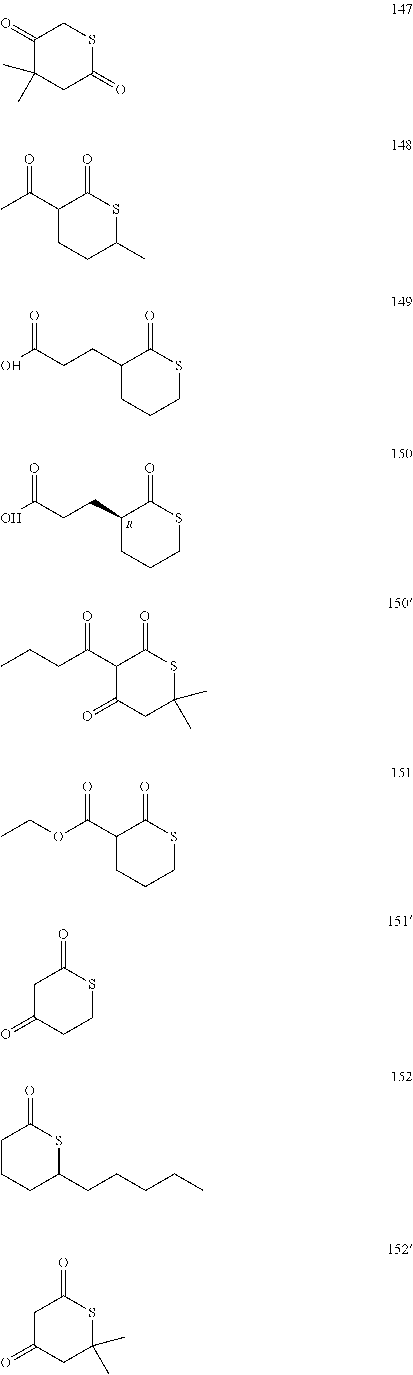

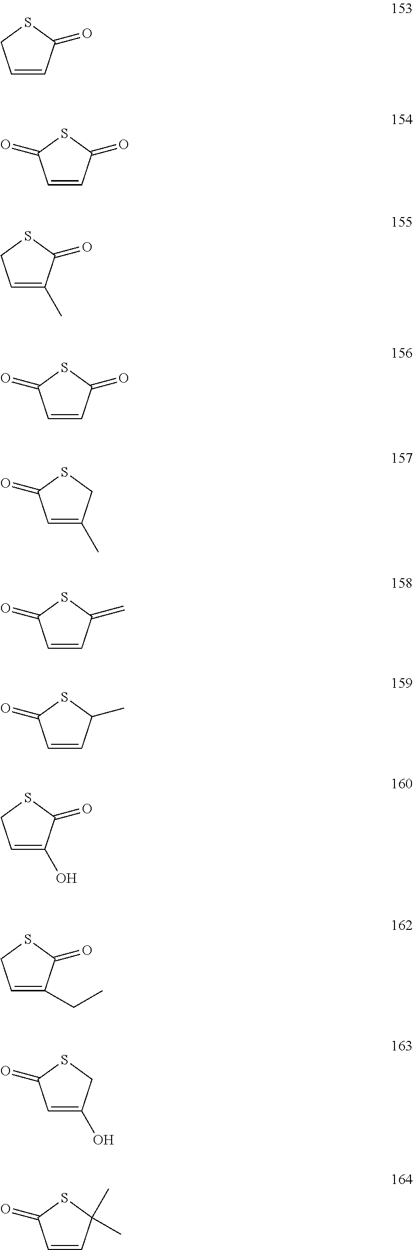

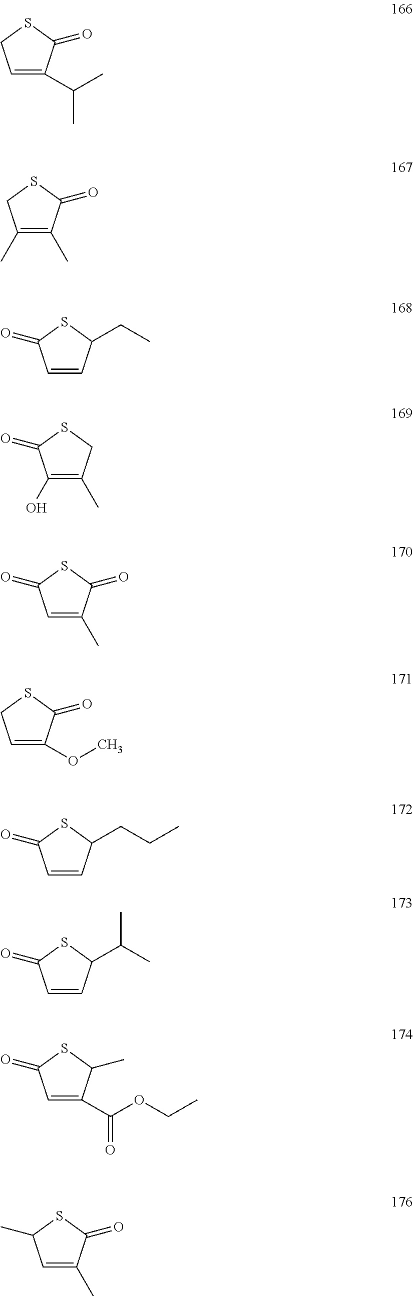

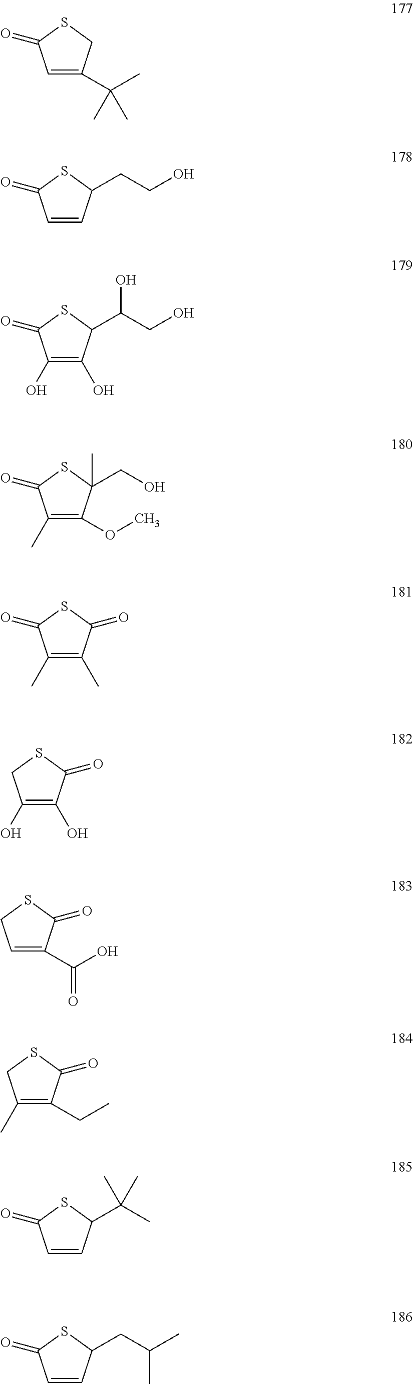

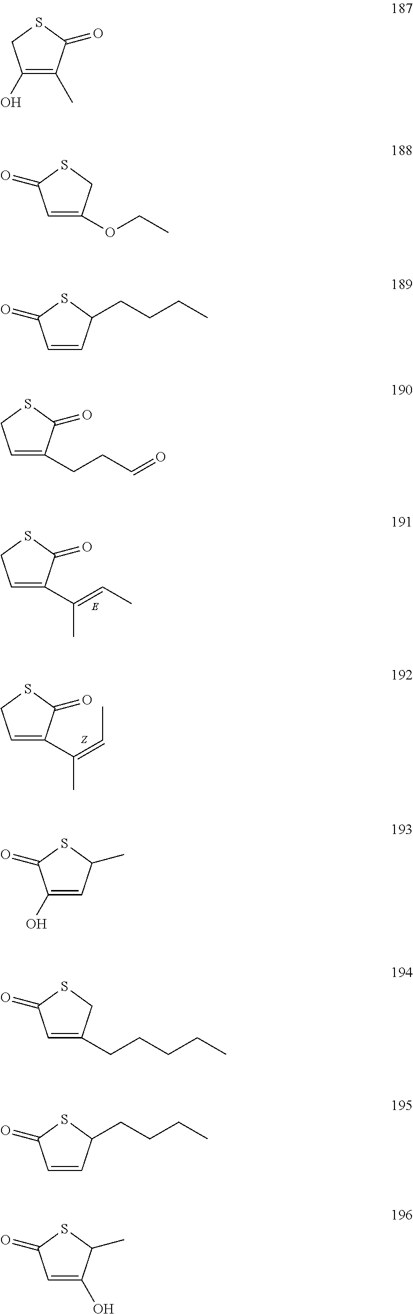

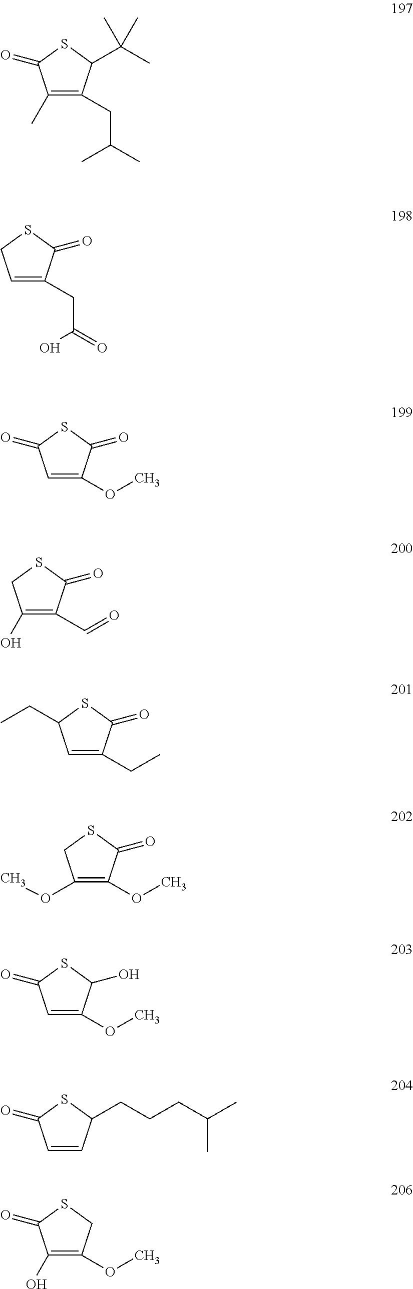

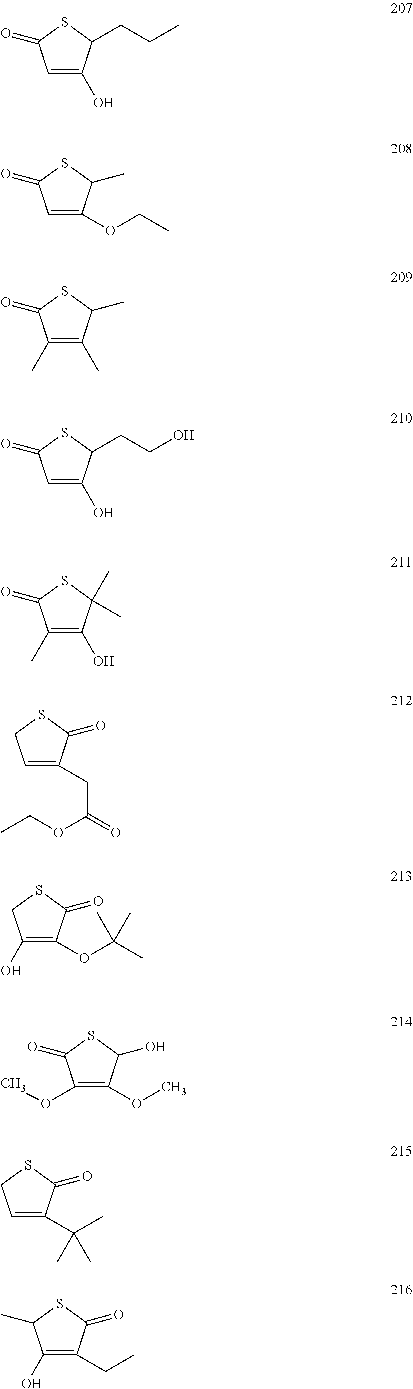

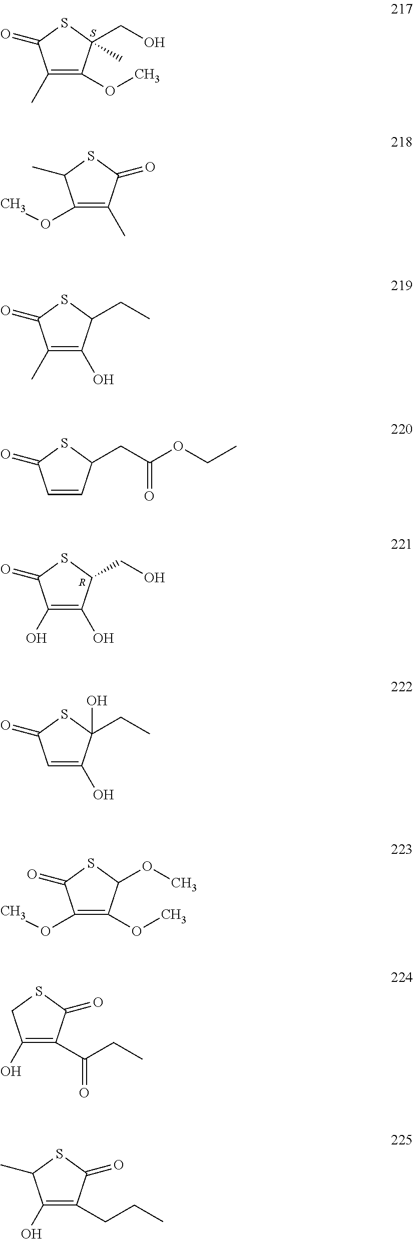

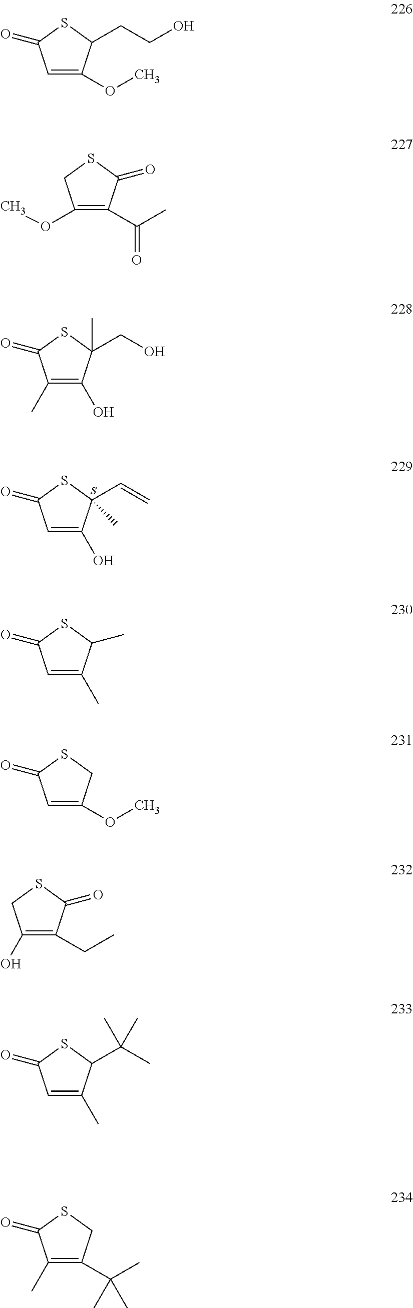

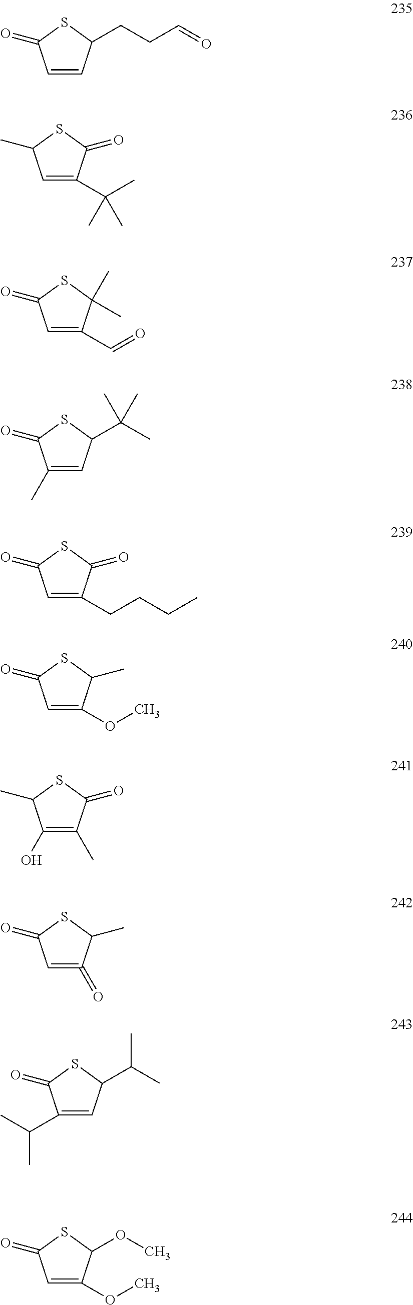

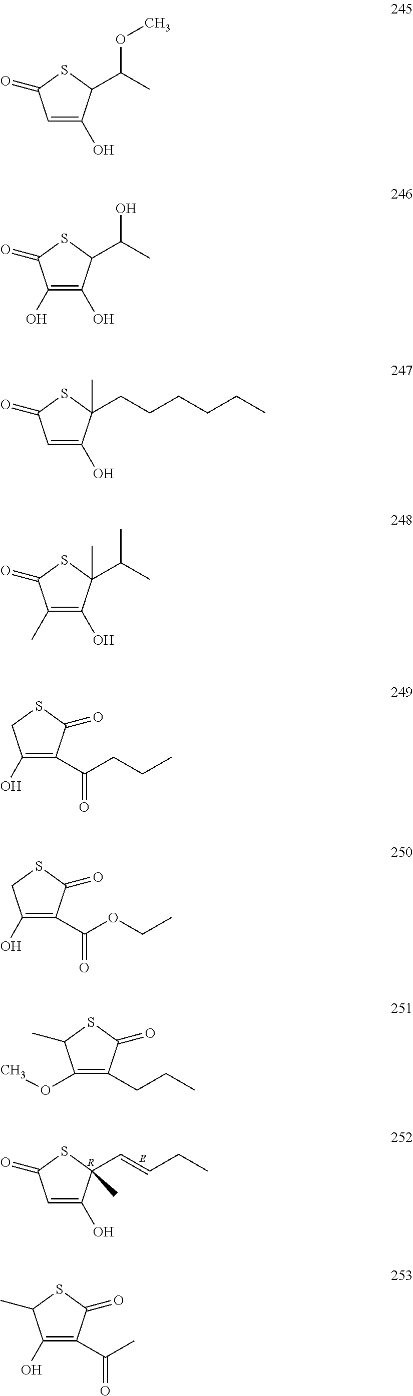

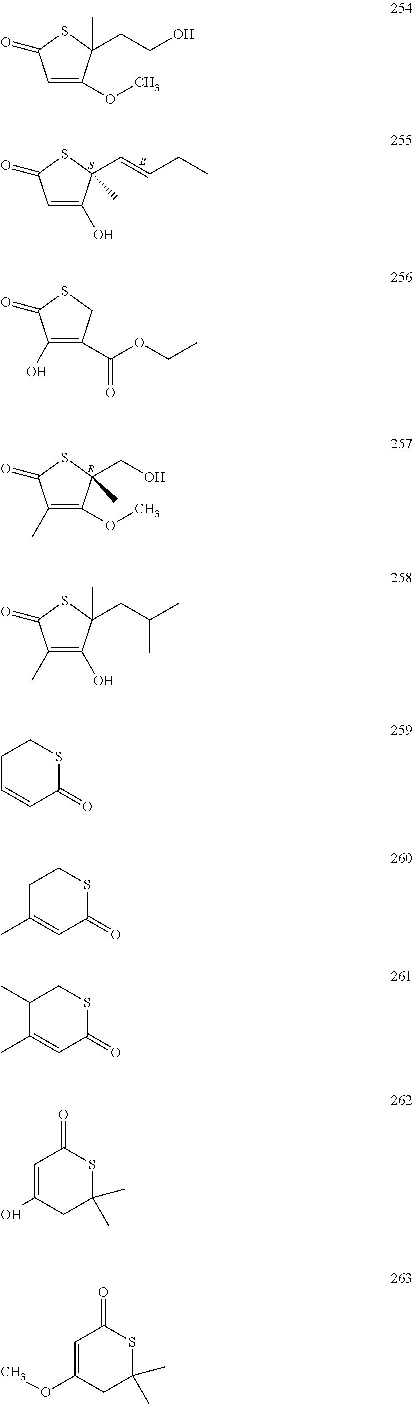

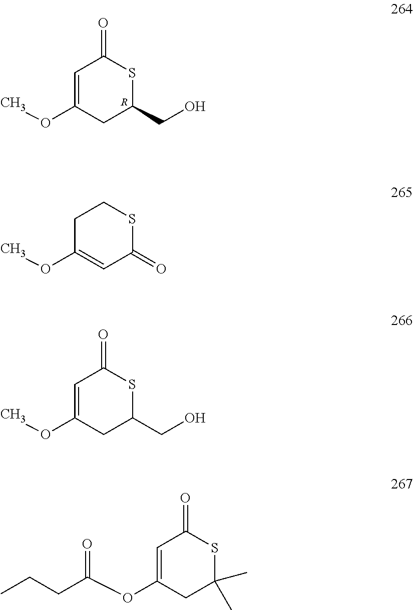

and also the organic or mineral acid or base salts thereof, the optical isomers thereof, the geometrical isomers thereof, the tautomers thereof, and the solvates thereof such as hydrates.

Preferably, the compound(s) of formula (I) is (are) chosen from the compounds of formulae (38) to (122) and (153) to (258), preferably from the compounds of formulae (38) to (42b), (70) to (71), (86) to (87), (90) to (95), (153) to (163), more preferably from the compounds of formulae (38), (42a), (42b), (71), (82), (89), (107′), (153), (163).

The process for preparing the compound of formula (I′b) is defined according to the following synthesis scheme:

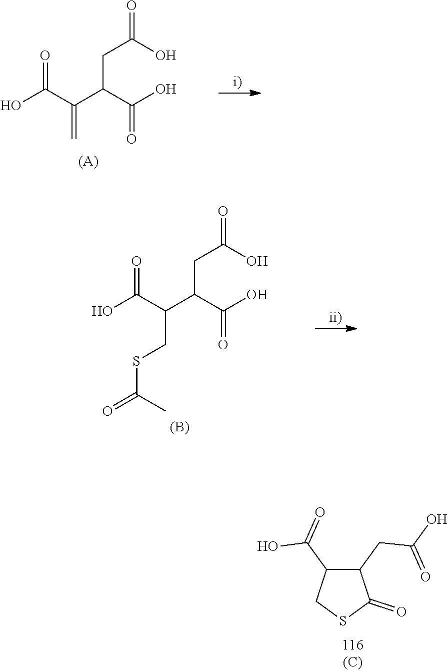

which consists in reacting, in step i), the 3-butene-1,2,3-tricarboxylic acid (A) with at least one molar equivalent of thioacetic acid, in particular in a non-protic organic solvent, which is preferably halogenated, such as dichloromethane, or ether of THF type, more particularly by heating the reaction medium up to the reflux of the solvent, preferably at a temperature of between 40° C. and 80° C., so as to give the compound (B); in step ii), the compound (B) reacts with an organic or inorganic strong acid such as hydrochloric acid, in particular in a polar protic solvent such as water, more particularly by heating at the reflux of the solvent, preferably at a temperature between 60 and 90° C., so as to give the thiolactone (C).

According to a particular embodiment, the composition comprises one or more compound(s) of formula (I) as defined previously, in an amount inclusively between 0.01% and 50%, in particular between 0.1% and 30%, more particularly between 1% and 25%, preferentially between 2% and 20%, more preferentially between 5% and 15% by weight relative to the total weight of the composition.

The composition of the invention also contains one or more silicones.

As silicones that may be used, mention may be made, alone or as a mixture, of polydialkylsiloxanes and especially polydimethylsiloxanes (PDMSs), polydiarylsiloxanes, polyalkylarylsiloxanes, silicone gums and resins, and also organopolysiloxanes (or organomodified polysiloxanes, or alternatively organomodified silicones) which are polysiloxanes comprising in their structure one or more organofunctional groups, generally attached via a hydrocarbon-based group, and preferably chosen from aryl groups, amino groups, hydroxyl groups, alkoxy groups and polyoxyethylene and/or polyoxypropylene groups.

The organomodified silicones may be polydiarylsiloxanes, in particular polydiphenylsiloxanes, and polyalkylarylsiloxanes, functionalized with the organofunctional groups mentioned previously. The polyalkylarylsiloxanes are particularly chosen from linear and/or branched polydimethyl/methylphenylsiloxanes and polydimethyl/diphenylsiloxanes.

Among the organomodified silicones, mention may be made of organopolysiloxanes comprising:
polyoxyethylene and/or polyoxypropylene groups optionally comprising C6-C24 alkyl groups, such as dimethicone copolyols, and especially those sold by the company Dow Corning under the name DC 1248 or the oils Silwet® L 722, L 7500, L 77 and L 711 from the company Union Carbide; or alternatively (C12)alkylmethicone copolyols, and especially those sold by the company Dow Corning under the name Q2 5200;
substituted or unsubstituted amine groups, in particular C1-C4 aminoalkyl groups; mention may be made of the products sold under the name GP4 Silicone Fluid and GP7100 by the company Genesee, or under the names Q2-8220 and DC929 or DC939 by the company Dow Corning;
thiol groups, such as the products sold under the names GP 72 A and GP 71 from Genesee;
alkoxylated groups, such as the product sold under the name Silicone Copolymer F-755 by SWS Silicones and Abil Wax® 2428, 2434 and 2440 by the company Goldschmidt;
hydroxylated groups, for instance polyorganosiloxanes bearing a hydroxyalkyl function;
acyloxyalkyl groups, such as the polyorganosiloxanes described in patent U.S. Pat. No. 4,957,732;
anionic groups of the carboxylic acid type, as described, for example, in EP 186 507, or of the alkylcarboxylic type, such as the product X-22-3701 E from the company Shin-Etsu; or alternatively of the 2-hydroxyalkylsulfonate or 2-hydroxyalkylthiosulfate type, such as the products sold by the company Goldschmidt under the names Abil® S201 and Abil® S255;
hydroxyacylamino groups, such as the polyorganosiloxanes described in patent application EP 342 834; mention may be made, for example, of the product Q2-8413 from the company Dow Corning.

The silicones may also be chosen from polydialkylsiloxanes, among which mention may be made mainly of polydimethylsiloxanes bearing trimethylsilyl end groups. Among these polydialkylsiloxanes, mention may be made of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from Dow Corning, such as DC200, with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

In this category of polydialkylsiloxanes, mention may also be made of the products sold under the names Abil Wax® 9800 and 9801 by the company Goldschmidt, which are poly(C₁-C₂₀)dialkylsiloxanes.

Products that may be used more particularly in accordance with the invention are mixtures such as:
mixtures formed from a polydimethylsiloxane with a hydroxy-terminated chain, or dimethiconol (CTFA), and from a cyclic polydimethylsiloxane, also known as cyclomethicone (CTFA), such as the product Q2-1401 sold by the company Dow Corning.

The polyalkylarylsiloxanes are chosen particularly from linear and/or branched polydimethyl/methylphenylsiloxanes and polydimethyl/diphenylsiloxanes with a viscosity of from 1×10⁻⁵ to 5×10⁻² m²/s at 25° C.

Among these polyalkylarylsiloxanes, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

According to one preferred embodiment, the composition comprises one or more functionalized silicone(s) comprising at least one functional group preferably chosen from amino groups, alkoxy groups and hydroxyl groups.

Preferably, the composition according to the invention comprises one or more amino silicones.

The term “amino silicone” is intended to mean any silicone comprising at least one primary, secondary or tertiary amine function or a quaternary ammonium group.

The composition according to the invention preferably comprises one or more amino silicones. The term “amino silicone” denotes any silicone comprising at least one primary, secondary or tertiary amine or a quaternary ammonium group.

The weight-average molecular masses of these amino silicones may be measured by gel permeation chromatography (GPC) at ambient temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

Preferably, the amino silicone(s) that may be used in the context of the invention are chosen from:

a) the polysiloxanes corresponding to formula (A):

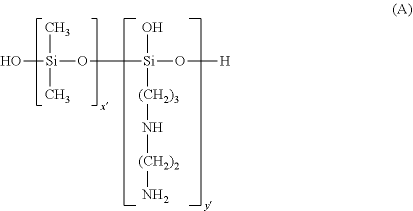

in which x′ and y′ are integers such that the weight-average molecular weight (Mw) is between 5000 and 500 000 approximately;

b) the amino silicones corresponding to formula (B):

R′ₐG₃₋ₐ-Si(OSiG₂)ₙ-(OSiG_{b}R′_{2-b})ₘ—O-SiG₃₋ₐ-R′ₐ (B)

in which:
G, which may be identical or different, denotes a hydrogen atom or a phenyl, OH or C₁-C₈ alkyl, for example methyl, or C₁-C₈ alkoxy, for example methoxy, group,
a, which may be identical or different, denotes 0 or an integer from 1 to 3, in particular 0,
b denotes 0 or 1, in particular 1,
m and n are numbers such that the sum (n+m) ranges from 1 to 2000 and in particular from 50 to 150, n possibly denoting a number from 0 to 1999 and in particular from 49 to 149, and m possibly denoting a number from 1 to 2000 and in particular from 1 to 10;
R′, which may be identical or different, denotes a monovalent radical of formula —C_{q}H_{2q}L in which q is a number ranging from 2 to 8 and L is an optionally quaternized amino group chosen from the following groups:
—N(R″)₂; —N+(R″)₃ A-; —NR″-Q-N(R″)₂ and —NR″-Q-N+(R″)₃ A-,

in which R″, which may be identical or different, denotes hydrogen, phenyl, benzyl, or a saturated monovalent hydrocarbon-based radical, for example a C1-C20 alkyl radical; Q denotes a linear or branched group of formula CᵣH₂ᵣ, r being an integer ranging from 2 to 6, preferably from 2 to 4; and A- represents a cosmetically acceptable anion, in particular a halide such as fluoride, chloride, bromide or iodide.

Preferably, the amino silicones are chosen from the amino silicones of formula (B). Preferably, the amino silicones of formula (B) are chosen from the amino silicones corresponding to formulae (C), (D), (E), (F) and/or (G) below.

According to a first embodiment, the amino silicones corresponding to formula (B) are chosen from the silicones known as “trimethylsilyl amodimethicone” corresponding to formula (C):

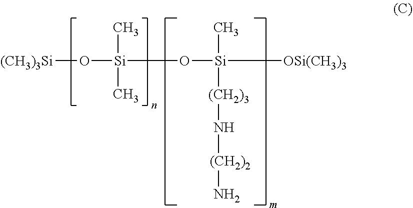

in which m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10.

According to a second embodiment, the amino silicones corresponding to formula (B) are chosen from the silicones of formula (D) below:

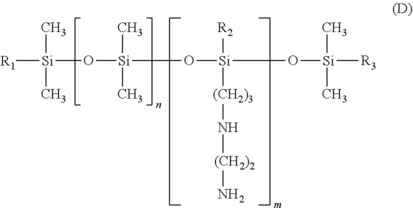

in which:
m and n are numbers such that the sum (n+m) ranges from 1 to 1000 and in particular from 50 to 250 and more particularly from 100 to 200; it being possible for n to denote a number from 0 to 999 and in particular from 49 to 249 and more particularly from 125 to 175, and form to denote a number from 1 to 1000 and in particular from 1 to 10, and more particularly from 1 to 5;
R1, R2 and R3, which may be identical or different, represent a hydroxyl or C1-C4 alkoxy radical, at least one of the radicals R₁ to R₃ denoting an alkoxy radical.

Preferably, the alkoxy radical is a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular mass (Mw) of these silicones preferably ranges from 2000 to 1 000 000 and more particularly from 3500 to 200 000.

According to a third embodiment, the amino silicones corresponding to formula (B) are chosen from the silicones of formula (E) below:

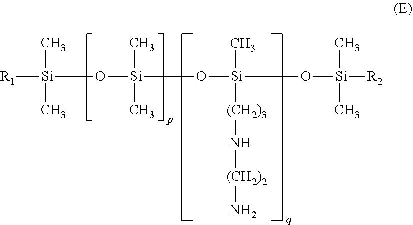

in which:
p and q are numbers such that the sum (p+q) ranges from 1 to 1000, in particular from 50 to 350 and more particularly from 150 to 250; it being possible for p to denote a number from 0 to 999 and in particular from 49 to 349 and more particularly from 159 to 239, and for q to denote a number from 1 to 1000, in particular from 1 to 10 and more particularly from 1 to 5;
R1 and R2, which are different, represent a hydroxyl or C1-C4 alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly equals 1:0.95.

The weight-average molecular mass (Mw) of the silicone preferably ranges from 2000 to 200 000, even more particularly from 5000 to 100 000 and more particularly from 10 000 to 50 000.

The commercial products comprising silicones of structure (D) or (E) may include in their composition one or more other amino silicones of which the structure is different from formula (D) or (E).

A product containing amino silicones of structure (D) is sold by the company Wacker under the name Belsil® ADM 652.

A product containing amino silicones of structure (E) is sold by Wacker under the name Fluid WR 1300®.

Among the amino silicones of formula (E), mention may also be made of the product Belsil ADM Log 1 from Wacker.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or non-ionic. The numerical mean size of the silicone particles in the emulsion generally ranges from 3 nm to 500 nm. Preferably, in particular as amino silicones of formula (E), use is made of microemulsions of which the mean particle size ranges from 5 nm to 60 nm (limits included) and more particularly from 10 nm to 50 nm (limits included). Thus, use may be made according to the invention of the amino silicone microemulsions of formula (E) sold under the names Finish CT 96 E® or SLM 28020® by the company Wacker.

According to a fourth embodiment, the amino silicones corresponding to formula (B) are chosen from the silicones of formula (F) below:

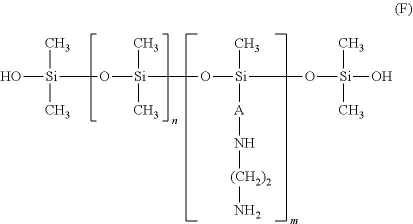

in which:
m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular mass (Mw) of these amino silicones preferably ranges from 2000 to 1 000 000 and even more particularly from 3500 to 200 000.

A silicone corresponding to this formula is, for example, the Xiameter MEM 8299 Emulsion from Dow Corning.

According to a fifth embodiment, the amino silicones corresponding to formula (B) are chosen from the silicones of formula (G) below:

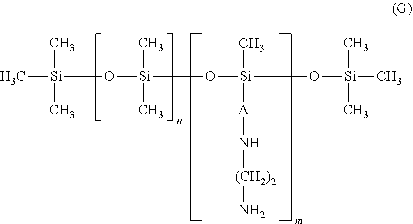

in which:
m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular mass (Mw) of these amino silicones preferably ranges from 500 to 1 000 000 and even more particularly from 1000 to 200 000.

A silicone corresponding to this formula is, for example, DC2-8566 Amino Fluid from Dow Corning.

c) the amino silicones corresponding to formula (H):

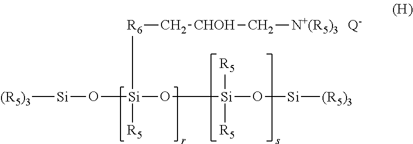

in which:
R₅ represents a monovalent hydrocarbon-based radical containing from 1 to 18 carbon atoms, and in particular a C₁-C₁₈ alkyl or C₂-C₁₈ alkenyl, for example methyl, radical;
R₆ represents a divalent hydrocarbon-based radical, in particular a C₁-C₁₈ alkylene radical or a divalent C₁-C₁₈, for example C₁-C₈, alkyleneoxy radical linked to the Si via an SiC bond;
Q⁻ is an anion such as a halide, especially chloride, ion or an organic acid salt, especially acetate;
r represents a mean statistical value ranging from 2 to 20 and in particular from 2 to 8;
s represents a mean statistical value ranging from 20 to 200 and in particular from 20 to 50.

Such amino silicones are in particular described in patent U.S. Pat. No. 4,185,087;

d) the quaternary ammonium silicones of formula (I):

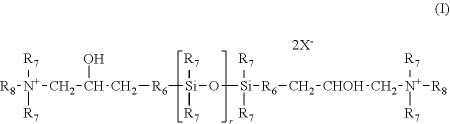

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example a methyl radical;
R₆ represents a divalent hydrocarbon-based radical, in particular a C₁-C₁₈ alkylene radical or a divalent C₁-C₁₈, for example C₁-C₈, alkyleneoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X⁻ is an anion such as a halide, especially chloride, ion or an organic acid salt, especially acetate;
r represents a mean statistical value ranging from 2 to 200 and in particular from 5 to 100.

These silicones are described, for example, in patent application EP-A 0 530 974.

e) the amino silicones of formula (J):

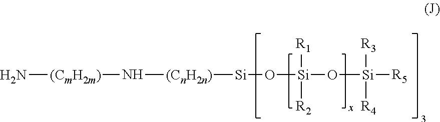

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g.

f) multiblock polyoxyalkylenated amino silicones, of the type (AB)n, A being a polysiloxane block and B being a polyoxyalkylene block comprising at least one amine group.

Said silicones preferably are constituted of repeating units of the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which may be identical or different, represent a linear or branched divalent C₂-C₁₂ hydrocarbon-based radical, optionally comprising one or more heteroatoms such as oxygen; preferably, R denotes an ethylene radical, a linear or branched propylene radical, a linear or branched butylene radical or a radical —CH₂CH₂CH₂OCH₂CH(OH)CH₂—; preferentially, R denotes a radical —CH₂CH₂CH₂OCH(OH)CH₂—;
R′, which may be identical or different, represent a linear or branched divalent C₂-C₁₂ hydrocarbon-based radical, optionally comprising one or more heteroatoms such as oxygen; preferably, R′ denotes an ethylene radical, a linear or branched propylene radical, a linear or branched butylene radical or a radical —CH₂CH₂CH₂OCH₂CH(OH)CH₂—; preferentially, R′ denotes —CH(CH₃)—CH₂—.

The siloxane blocks preferably represent 50 mol % to 95 mol % of the total weight of the silicone, more particularly from 70 mol % to 85 mol %.

The amine content is preferably between 0.02 and 0.5 meq./g of copolymer in a 30% solution in dipropylene glycol, more particularly between 0.05 and 0.2.

The weight-average molecular mass (Mw) of the silicone is preferably between 5000 and 1 000 000 and more particularly between 10 000 and 200 000.

Mention may be made especially of the silicones sold under the names Silsoft A-843 or Silsoft A+ by Momentive.

g) and mixtures thereof.

Preferably, the amino silicone(s) that can be used in the composition according to the invention is (are) chosen from the amino silicones of formula (B) and the multiblock polyoxyalkylenated amino silicones, of the type (AB)n, more particularly from the amino silicones of formulae (C), (D), (E), (F) and (G) and the multiblock polyoxyalkylenated amino silicones.

Even more preferably, the amino silicone(s) that can be used in the composition according to the invention is (are) chosen from the amino silicones of formula (E) and the multiblock polyoxyalkylenated amino silicones.

According to one preferred embodiment, the composition comprises one or more silicones chosen from functionalized silicone(s) comprising at least one functional group chosen from amino groups and hydroxyl groups.

According to one particularly preferred embodiment, the composition comprises one or more silicones chosen from polydimethylsiloxanes comprising dimethylsilanol end groups, the amino silicones of formula (E) and the multiblock polyoxyalkylenated amino silicones.

According to one particular embodiment, the composition comprises one or more silicone(s), preferably amino silicone(s), as defined previously, in an amount inclusively between 0.01% and 10%, in particular between 0.1% and 5%, more particularly between 0.5% and 5% by weight relative to the total weight of the composition.

According to one particular embodiment of the invention, the composition comprises a thiol-comprising a reducing agent such as thioglycolic acid, or a non-thiol-comprising reducing agent.

Preferentially, the composition according to the invention is aqueous.

Preferentially, the composition according to the invention comprises water at a concentration ranging from 10% to 99.5%, better still from 30% to 95% and even better still from 40% to 90% by weight relative to the total weight of the composition.

The pH of the composition of the invention may be less than or equal to 7. Preferably, the pH of the composition ranges from 1 to 6 and more preferably from 2 to 6.

The pH values may be adjusted with an organic or mineral acid, or with an alkaline agent chosen from mineral or organic or hybrid alkaline agents or mixtures thereof.

The term “organic acid” is intended to mean an acid, i.e. a compound that is capable of releasing a cation or proton H⁺ or H₃O⁺, in aqueous medium, which comprises at least one optionally unsaturated, linear or branched C₁-C₂₀ hydrocarbon-based chain, a (hetero)cycloalkyl or (hetero)aryl group and at least one acidic chemical function chosen in particular from carboxyl C(O)OH, sulfuric SO₃H, SO₂H, and phosphoric PO₃H₂, PO₄H₂.

More particularly, the acids used are chosen from hydrochloric acid HCl, hydrobromic acid HBr, sulfuric acid H₂SO₄, alkylsulfonic acids: (C₁-C₆)Alk-S(O)₂OH such as methanesulfonic acid and ethanesulfonic acid; arylsulfonic acids: Ar—S(O)₂OH such as benzenesulfonic acid and toluenesulfonic acid; (C₁-C₆)alkoxysulfinic acids: Alk-O—S(O)OH such as methoxysulfinic acid and ethoxysulfinic acid; aryloxysulfinic acids such as tolueneoxysulfinic acid and phenoxysulfinic acid; phosphoric acid H₃PO₄; triflic acid CF₃SO₃H and tetrafluoroboric acid HBF₄, and carboxylic acid(s) of formula (K) below:

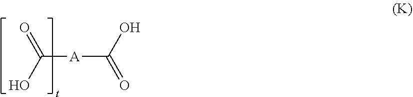

in which formula (K) or a salt thereof:
A represents a saturated or unsaturated, cyclic or non-cyclic, and aromatic or non-aromatic hydrocarbon-based group, which is monovalent when t is 0 or polyvalent when t is greater than or equal to 1, comprising from 1 to 50 carbon atoms, which is optionally interrupted with one or more heteroatoms and/or optionally substituted, especially with one or more hydroxyl groups; preferably, A represents a monovalent (C₁-C₆)alkyl group or a polyvalent (C₁-C₆)alkylene group optionally substituted with one or more hydroxyl groups.

Particularly, the carboxylic acid(s) of formula (K) as defined previously, and preferably the acid(s) used, is/are an alpha-hydroxy acid such as lactic acids, glycolic acids, tartaric acids or citric acids.

The mineral alkaline agent(s) are preferably chosen from aqueous ammonia, alkaline carbonates or bicarbonates such as sodium or potassium carbonates and sodium or potassium bicarbonates, sodium hydroxide or potassium hydroxide, or mixtures thereof.

According to an advantageous embodiment of the invention, the alkaline agent(s) are organic amines, i.e. they contain at least one substituted or unsubstituted amino group.

The organic alkaline agent(s) are more preferentially chosen from organic amines with a pK_{b} at 25° C. of less than 12, preferably of less than 10 and even more advantageously of less than 6. It should be noted that it concerns the pK_{b} corresponding to the function having the highest basicity.

Hybrid compounds that may be mentioned include the salts of the amines mentioned previously with acids such as carbonic acid or hydrochloric acid.

The organic alkaline agent(s) are chosen, for example, from alkanolamines, oxyethylenated and/or oxypropylenated ethylenediamines, amino acids and the compounds of formula (L) below:

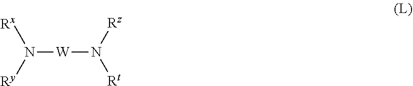

in which formula (L):
W is a divalent C₁-C₆ alkylene radical optionally substituted with a hydroxyl group or a C₁-C₆ alkyl radical, and/or optionally interrupted with one or more heteroatoms such as oxygen or NR^{u};
R^{x}, R^{y}, R^{z}, R^{t} and R^{u}, which may be identical or different, represent a hydrogen atom or a C₁-C₆ alkyl, C₁-C₆ hydroxyalkyl or C₁-C₆ aminoalkyl radical.

Preferably, the alkanolamine is ethanolamine (or monoethanolamine).

In one variant of the invention, the composition comprises, as alkaline agent, one or more alkanolamines (preferably ethanolamine) and aqueous ammonia. In this variant, the alkanolamine(s) are present in a predominant amount relative to the aqueous ammonia.

The composition of the invention can also comprise at least one usual cosmetic ingredient, in particular chosen from surfactants, in particular non-ionic or cationic surfactants, solid or liquid fatty substances, thickeners, in particular polysaccharide thickeners, non-silicone cationic polymers, silicones other than the amino silicones, polar or non-polar solvents, sunscreens; moisturizers; antidandruff agents; antioxidants; chelating agents; pearlescent agents and opacifiers; plasticizers and coalescence agents; fillers; fragrances; silanes; and crosslinking agents. The composition can, of course, comprise several cosmetic ingredients appearing in the above list.

Depending on their nature and the purpose of the composition, the usual cosmetic ingredients may be present in usual amounts, which can be readily determined by those skilled in the art and which may be, for each ingredient, between 0.01% and 80% by weight. Those skilled in the art will take care to select the ingredients included in the composition, and also the amounts thereof, so that they do not harm the properties of the compositions of the present invention.

The compositions used in the process according to the invention may be in any formulation form conventionally used, and especially in the form of an aqueous, alcoholic or aqueous-alcoholic, or oily, solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

The composition which has just been described can be applied to the hair. The bath ratio for the composition applied, that is to say the amount by weight of composition applied relative to the weight of hair treated, can range from 0.1 to 10, more particularly from 0.2 to 5, and preferably is between 0.5 and 3. The term “bath ratio” is intended to mean the ratio between the total weight of the applied composition and the total weight of keratin fibres to be treated.

The composition of the invention may be applied to dry or wet keratin materials, preferably to dry or wet hair, preferably to dry hair.

In particular, the step of applying the composition may be followed by a leave-on time. The leave-on time, namely the time of contact of the composition on the hair, is preferably at least 5 minutes, preferably between 10 and 60 minutes and preferably between 15 and 45 minutes. Rinsing of the hair may optionally be envisaged after the application of the composition and optionally the leave-on time.

The hair may then optionally be wrung dry, preferably wrung dry.

As previously indicated, the step of applying the composition which has been described is followed by a hair shaping step.

The shaping step can be a straightening/relaxing step or else a step for combing the hair.

This shaping step can be carried out by means of curlers, a curling iron or a straightening iron (also called flat tongs).

When the shaping step is carried out by means of an iron, it can be carried out at a temperature of at least 100° C.

Preferably, the iron is used at a temperature of at least 100° C., preferably at a temperature between, limits included, 100° C. and 300° C., preferably between 120° C. and 280° C., more preferably between 150° C. and 250° C., and better still between 200 and 250° C.

According to one particular embodiment, a straightening iron is used. In this embodiment, the straightening with the straightening iron is performed in several passes on the hair, in general 3 to 10 passes.

According to a particular embodiment, the process of the invention comprising the steps of applying the composition according to the invention to the hair, then of straightening with an iron, is performed one or more times, optionally separated by one or more cosmetic treatments, preferably a shampooing operation, until the desired shape or shape intensity is obtained.

According to one particular embodiment of the invention, a composition comprising an oxidizing agent can be applied to the hair, after the shaping step. This oxidation step can be followed by rinsing of the hair.

According to a particular embodiment of the invention, the process for treating keratin materials of the invention does not include any thiol-based reducing agent such as thiolactic acid; preferably, the process does not include any reducing agent.

According to a particular embodiment, the process for treating keratin materials does not include a lanthionization step. Preferably, the process for treating keratin fibres does not include an alkaline agent.

The examples that follow serve to illustrate the invention without, however, being limiting in nature.

### EXAMPLES

The following compositions were prepared from the ingredients indicated in the tables below, all the amounts being indicated as percent by weight of active material, relative to the total weight of the composition.

Examples: compositions T1 to T6 for comparative controls

| | T1 | T2 | T3 | T4 | T5 | T6 |
|---|---|---|---|---|---|---|
| Thiolactone 38 | 5 | | | | | |
| Ethanol | 30 | 30 | 30 | 30 | 30 | 30 |
| Amodimethicone/trideceth-5/trideceth-10* | - | - | 5 | - | 1 | 1 |
| PEG-40/PPG-8 | - | 5 | - | 2 | - | 2 |
| Methylaminopropyl/hydroxypropyl | | | | | | |
| dimethicone copolymer** | | | | | | |
| Water (qs) | 100 | 100 | 100 | 100 | 100 | 100 |

*Belsil ADM LOG 1 from Wacker
**Silsoft A+ LT from Momentive

Examples: compositions 1 to 9 according to the invention

| | 1 | 2 | 3 | 4 | 5 | 6 | 7 | 8 | 9 |
|---|---|---|---|---|---|---|---|---|---|
| Thiolactone 38 | 5 | 5 | 10 | 10 | 10 | 10 | 10 | 15 | 10 |
| Ethanol | 30 | 30 | 30 | 30 | 30 | 30 | 30 | 30 | 30 |
| Amodimethicone/ | - | 5 | - | 1 | 1 | 1 | 1 | 1 | 1 |
| trideceth-5/trideceth-10* | | | | | | | | | |
| PEG-40/PPG-8 | 5 | - | 2 | - | 2 | 2 | 2 | 2 | 2 |
| Methylaminopropyl/hydroxypropyl | | | | | | | | | |
| dimethicone | | | | | | | | | |
| copolymer** | | | | | | | | | |
| Dimethyl sulfone | - | - | - | - | - | - | - | - | 5 |
| Triethanolamine qs pH: | - | - | - | - | - | - | - | 8 | - |
| Fragrance | - | - | - | - | - | - | 0.5 | 0.5 | 0.5 |
| Hydroxyethylcellulose*** | - | - | - | - | - | 0.75 | 0.75 | 0.75 | 1.00 |
| Water (qs) | 100 | 100 | 100 | 100 | 100 | 100 | 100 | 100 | 100 |

*Belsil ADM LOG 1 from Wacker
**Silsoft A+ LT from Momentive
***Natrosol 250 HHR CS from Ashland, MW 1 300 000

For each example, a lock of frizzy natural Caucasian hair of frizziness type IV, weighing 2.7 g having a length of 20 or 27 cm was treated in the following way:

The locks are wetted, a shampoo is applied (DOP camomille based on sodium laureth sulfate, on coco betaine, on glycerol and on cocamide MEA) in a proportion of 400 mg per lock. The locks are massaged and then rinsed with running water, wiped dry with a finger and dried with a hairdryer. The composition (from T1 to T6 for the controls, and from 1 to 9 according to the invention) is applied in a proportion of 2.7 g per lock, the lock is left in contact with the composition for 30 minutes, then it is dried with a hairdryer and straightened with flat tongs brought to 230° C. which are passed over each lock 10 times.

Each lock is then evaluated according to 3 main criteria for an indication of the performance level of the straightening, in the time:

I—Level of relaxation after 1 shampooing operation and natural drying

II—Level of relaxation after 5 shampooing cycles and natural drying
The level of relaxation is evaluated using a set of reference locks, with the following grades:
   1: no relaxation
   2: medium relaxation
   3: good level of relaxation
   4: very good level of relaxation

III—Volume of the lock after 1 shampooing operation

IV—Volume of the lock after 5 shampooing operations

The volume is evaluated using a set of reference locks, with the following grades:
1: large amount of volume
2: medium volume
3: little volume
4: no volume

V—Respect of lock integrity: smooth to the touch, general appearance, breakage during combing, appearance of the ends

VI—Cosmetic contribution: smooth feel, disentangling of wet or dry hair.

The results obtained for the controls are as follows:

| Example | T1 | T2 | T3 | T4 | T5 | T6 |
|---|---|---|---|---|---|---|
| Criterion I | 2 | 1 | 1 | 1 | 1 | 1 |
| Criterion II | 1 | 1 | 1 | 1 | 1 | 1 |
| Criterion III | 2 | 2.5 | 1 | 2.5 | 1 | 1 |
| Criterion IV | 1 | 2 | 1 | 2 | 1 | 1 |
| Criterion V | - | - | - | - | - | - |
| Criterion VI | -- | -- | -- | -- | -- | -- |

- integrity respected
-- natural feel

The results obtained for the compositions according to the invention are as follows:

| Example | 1 | 2 | 3 | 4 | 5 | 6 | 7 | 8 | 9 |
|---|---|---|---|---|---|---|---|---|---|
| Criterion I | 2.5 | 3 | 4 | 4 | 4 | 4 | 4 | 4 | 4 |
| Criterion II | 1.5 | 1.5 | 3 | 3 | 3 | 3 | 3 | 3 | 3 |
| Criterion III | 2.5 | 3.5 | 3.5 | 3.5 | 3.5 | 3.5 | 3.5 | 3.5 | 3.5 |
| Criterion IV | 2 | 1.5 | 3.5 | 3 | 3.5 | 3 | 3 | 3.5 | 3.5 |
| Criterion V | - | - | - | - | - | - | - | - | - |
| Criterion VI | * | ** | * | ** | *** | *** | *** | *** | *** |

- integrity of the locks respected
* hair smooth to the touch once dry; easier to pass over the flat tongs
** hair smooth to the touch while wet, easy to disentangle, after one shampooing operation and up to at least five shampooing operations
*** hair smooth to the touch once dry; easier to pass over the flat tongs; hair smooth to the touch while wet, easy to disentangle, after one shampooing operation and up to at least five shampooing operations

Moreover, locks having been bleached or dyed via a commercial oxidation colouring or relaxed locks are treated using the same protocol in order to evaluate the integrity and the latter is respected.

The composition according to the invention thus makes it possible to improve the relaxation of curls, to reduce the volume of the hair, and to improve the persistence of the relaxation and of the reduction in volume with respect to several shampooing operations, while at the same time keeping good integrity of the hair.

1-17. (canceled)

18. A composition comprising:
(a) at least one compound chosen from compounds of formula (I) below: 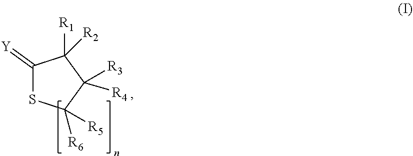
organic or mineral acid or base salts thereof, optical isomers thereof, geometrical isomers thereof, tautomers thereof, or solvates thereof;
wherein:
Y is chosen from an oxygen atom, a sulfur atom, or a group NR, wherein R is a hydrogen atom, a (C₁-C₆)alkyl group, or a hydroxy(C₁-C₆)alkyl group;
R₁, R₂, R₃, R₄, R₅ and R₆, which may be identical or different, are chosen from:
i) a hydrogen atom,
ii) hydroxyl,
iii) amino —NR′R″, wherein R′ and R″, which may be identical or different, are chosen from a hydrogen atom, a (C₁-C₆)alkyl, or a hydroxy(C₁-C₆)alkyl group;
iv) cyano,
v) —(Y′)ₚ—C(Y″)—(Y′″)_{q}—R₇, wherein R₇ is a hydrogen atom or a (C₁-C₆)alkyl group, p and q, which may be identical or different, are equal to 0 or 1, Y′, Y″ and Y″, which may be identical or different, are chosen from an oxygen atom, sulfur atom, or NR, wherein R is a hydrogen atom, a (C₁-C₆)alkyl group, or a hydroxy(C₁-C₆)alkyl group,
vi) optionally substituted (C₁-C₆)alkyl,
vii) optionally substituted (C₂-C₆)alkenyl,
viii) optionally substituted (C₁-C₆)alkoxy,
or alternatively:
ix) R₁ and R₃ together form a covalent bond;
x) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl optionally substituted with one or more hydroxyl groups, and d) (C₂-C₆)alkenyl, e) —(Y′)ₚ—C(Y″)—(Y′″)_{q}—R₇;
n represents an integer between 0 and 6 inclusive;
wherein:
when n is greater than or equal to 2, then the contiguous groups C(R₅)—R₆ may be identical or different;
and
(b) at least one silicone.

19. The composition according to claim 18, wherein the at least one compound of formula (I) is chosen from compounds of formula (Ia): 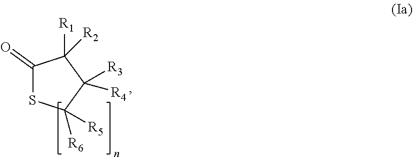
organic or mineral acid or base salts thereof, optical isomers thereof, geometrical isomers thereof, tautomers thereof, or solvates thereof;
wherein:
R₁, R₂, R₃, R₄, R₅, and R₆, which may be identical or different, are chosen from:
i) a hydrogen atom,
ii) hydroxyl,
iii) amino —NR′R″, with R′ and R″, which may be identical or different, chosen from a hydrogen atom, a (C₁-C₆)alkyl, or hydroxy(C₁-C₆)alkyl group;
iv) cyano,
v) —(Y′)ₚ—C(O)—(Y″)_{q}—R₇ with R₇ chosen from a hydrogen atom, a (C₁-C₆)alkyl group, p and q, which may be identical or different, being equal to 0 or 1, with Y′ and Y″, which may be identical or different, representing an oxygen atom or NR, wherein R is a hydrogen atom, a (C₁-C₆)alkyl group, or a hydroxy(C₁-C₆)alkyl group,
vi) (C₁-C₆)alkyl optionally substituted with one or more groups chosen from hydroxyl and —C(O)—R₈, wherein R₈ is chosen from a) a hydrogen atom, b) a hydroxyl group, or c) (C₁-C₆)alkoxy,
vii) (C₂-C₆)alkenyl,
viii) (C₁-C₆)alkoxy,
or
ix) R₁ and R₂, and/or R₃ and R₄, and/or R₅ and R₆ form, together with the carbon atom that bears them, a double bond oxo ═O or ═C(R₉)—R₁₀ wherein R₉ and R₁₀ are chosen from a) a hydrogen atom or a group chosen from b) hydroxyl, c) (C₁-C₆)alkyl.

20. The composition according to claim 19, wherein in the at least one compound of formula (Ia) is chosen from compounds: 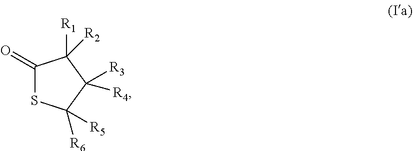
organic or mineral acid or base salts thereof, optical isomers thereof, geometrical isomers thereof, tautomers thereof, or solvates thereof;
wherein in formula (I′a):
R₁, R₂, R₃, R₄, R₅ and R₆, which may be identical or different, are chosen from:
i) a hydrogen atom,
ii) hydroxyl,
iii) amino —NR′R″, wherein R′ and R″, which may be identical or different, are chosen from a hydrogen atom, a (C₁-C₆)alkyl group, or a hydroxy(C₁-C₆)alkyl group;
iv) cyano,
v) —(Y′)ₚ—C(O)—(O)_{q}—R₇, wherein R₇ is chosen from a hydrogen atom or a (C₁-C₆)alkyl group, p and q, which may be identical or different, being equal to 0 or 1, with p+q=0 or 1, with Y′ representing an oxygen atom or NR, wherein R is chosen from a hydrogen atom, a (C₁-C₆)alkyl group, or a hydroxy(C₁-C₆)alkyl group;
vi) optionally substituted (C₁-C₆)alkyl,
vii) (C₂-C₆)alkenyl,
viii) (C₁-C₆)alkoxy,
wherein the alkyl groups are optionally substituted with one or more groups chosen from hydroxyl and —(O)ₜ—C(O)—R₈ wherein t is equal to 0 or 1, R₈ is chosen from a) a hydrogen atom, b) a hydroxyl group, c) (C₁-C₆)alkyl, d) (C₁-C₆)alkoxy, e) (di)(C₁-C₆)(alkyl)amino, or f) (di)hydroxy(C₁-C₆)alkylamino;
or alternatively:
ix) R₁ and R₂, and/or R₃ and R₄, and/or R₅ and R₆ form, together with the carbon atom that bears them, a double bond oxo ═O or ═C(R₉)—R₁₀ with R₉ and R₁₀ chosen from i) a hydrogen atom or a group chosen from ii) hydroxyl, iii) (C₁-C₆)alkyl optionally substituted with one or more hydroxyl groups.

21. The composition according to claim 18, wherein in the at least one compound of formula (I) is chosen from compounds of formula (Ic): 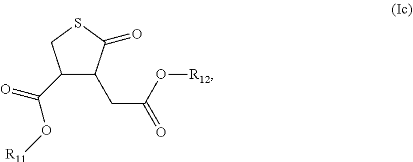
organic or mineral acid or base salts thereof, optical isomers thereof, geometrical isomers thereof, tautomers thereof, or solvates thereof;
wherein:
R₁₁ and R₁₂, which may be identical or different, are chosen from a hydrogen atom, a (C₁-C₆)alkyl group.

22. The composition according to claim 18, wherein in the at least one compound of formula (I), n is 1 or 2, and the compound is chosen from compounds of formula (Ib): 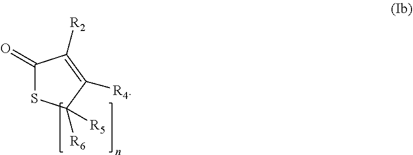
organic or mineral acid or base salts thereof, optical isomers thereof, geometrical isomers thereof, tautomers thereof, and solvates thereof;
wherein:
R₂, R₄, R₅ and R₆, which may be identical or different, are chosen from:
i) a hydrogen atom,
ii) hydroxyl,
iii) amino —NR′R″, R′ and R″, which may be identical or different, are chosen from a hydrogen atom, a (C₁-C₆)alkyl group, or a hydroxy(C₁-C₆)alkyl group;
iv) cyano,
v) —(Y′)ₚ—C(O)—(O)_{q}—R₇ wherein R₇ is chosen from a hydrogen atom or a (C₁-C₆)alkyl group, p and q, which may be identical or different, being equal to 0 or 1, with p+q=0 or 1, with Y′ representing an oxygen atom or NR wherein R is chosen from a hydrogen atom, a (C₁-C₆)alkyl group, or a hydroxy(C₁-C₆)alkyl group,
vi) (C₁-C₆)alkyl optionally substituted with one or more groups chosen from hydroxyl and —C(O)—R₈ wherein R₈ is chosen from a) a hydrogen atom, b) a hydroxyl group, or c) (C₁-C₆)alkoxy,
vii) (C₂-C₆)alkenyl,
viii) (C₁-C₆)alkoxy,
or alternatively:
ix) R₅ and R₆ form, together with the carbon atom that bears them, a double bond oxo ═O or ═C(R₉)—R₁₀ wherein R₉ and R₁₀ are chosen from a hydrogen atom or a (C₁-C₆)alkyl group.

23. The composition according to claim 20, wherein the at least one compound of formula (I) is chosen from compounds of formula (I′a) and n is 1.

24. The composition according to claim 18, wherein the at least one compound of formula (I) is chosen from the following compounds: 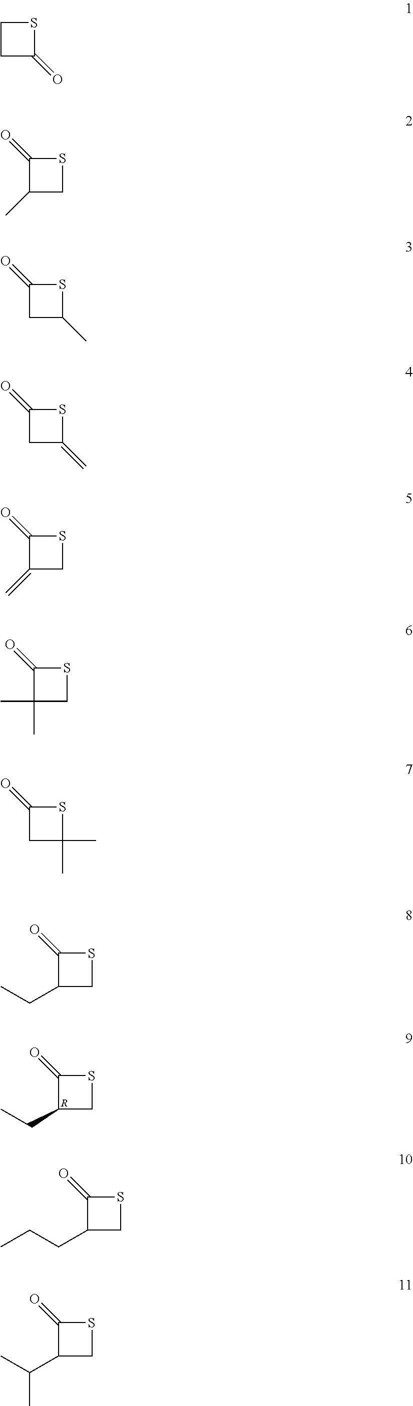 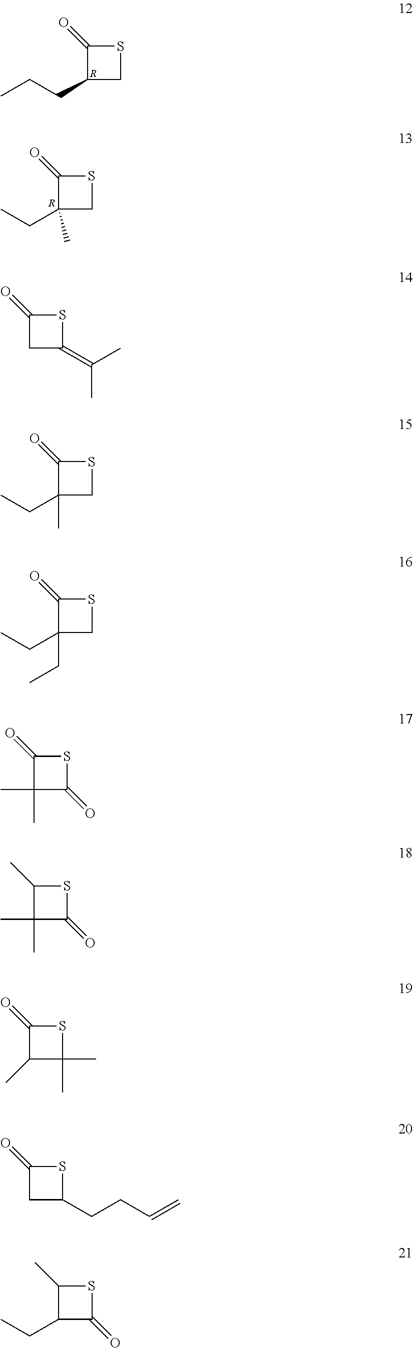 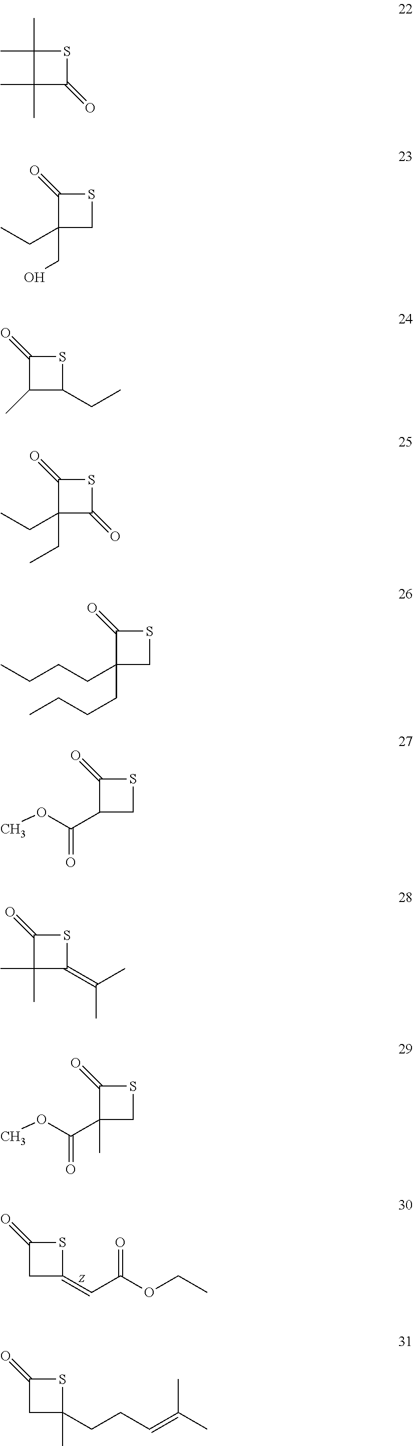 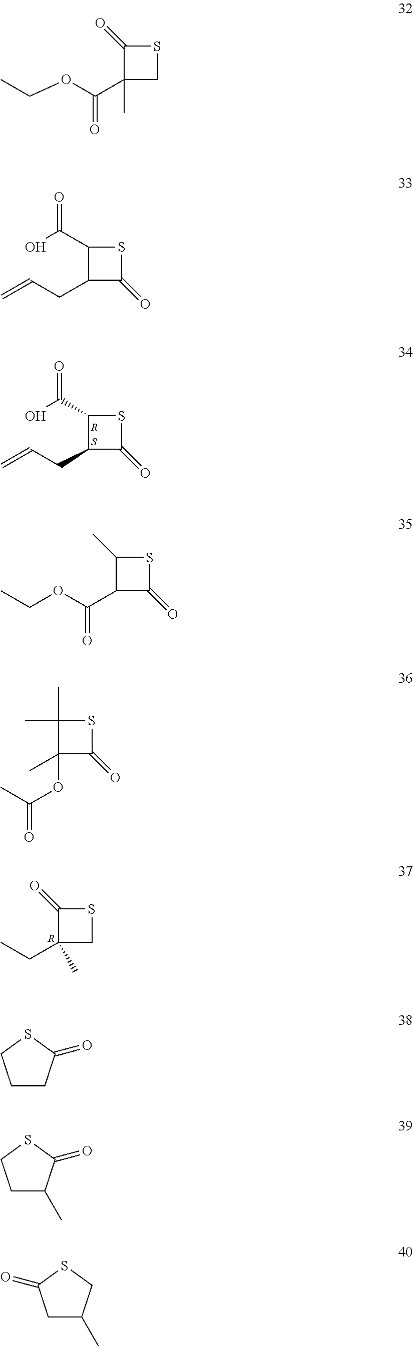 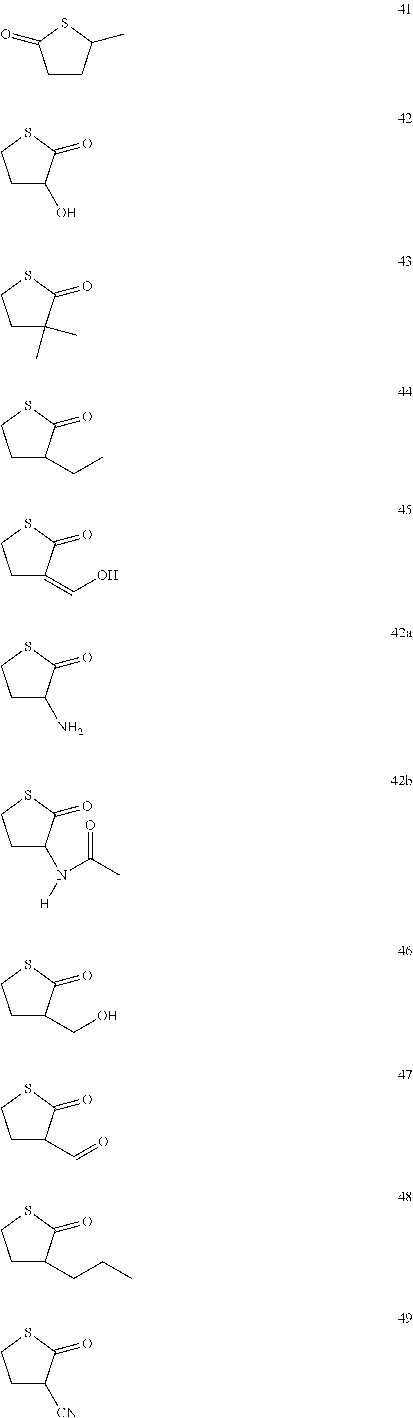 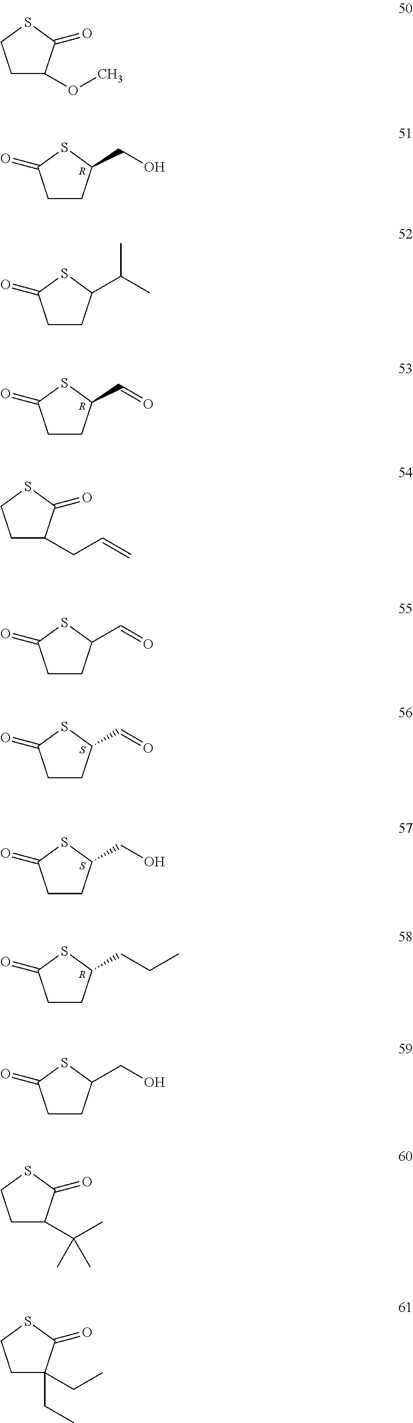 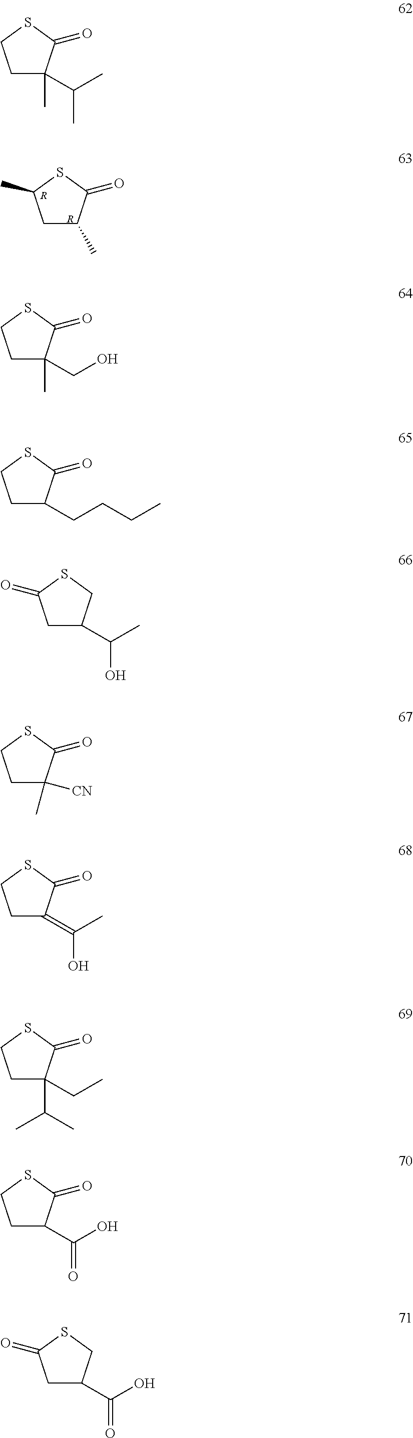 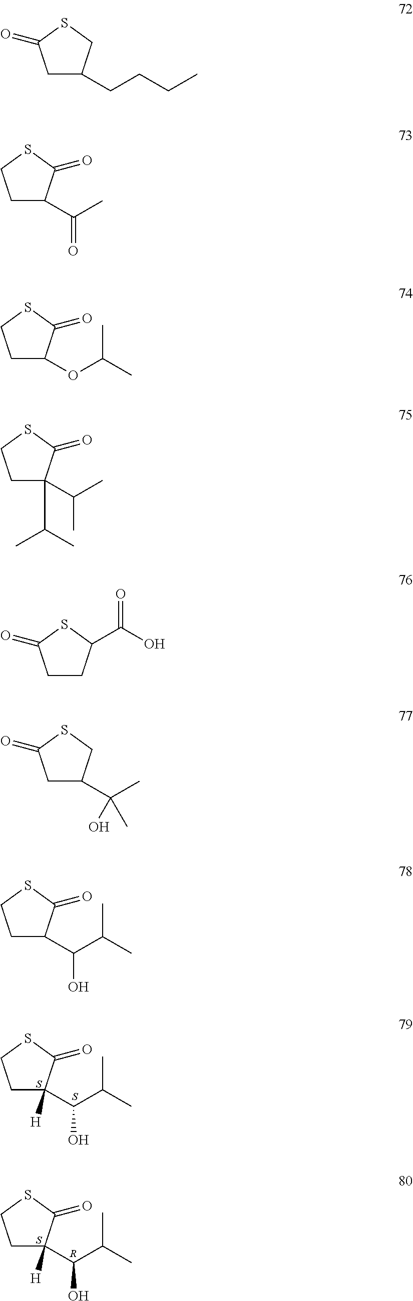 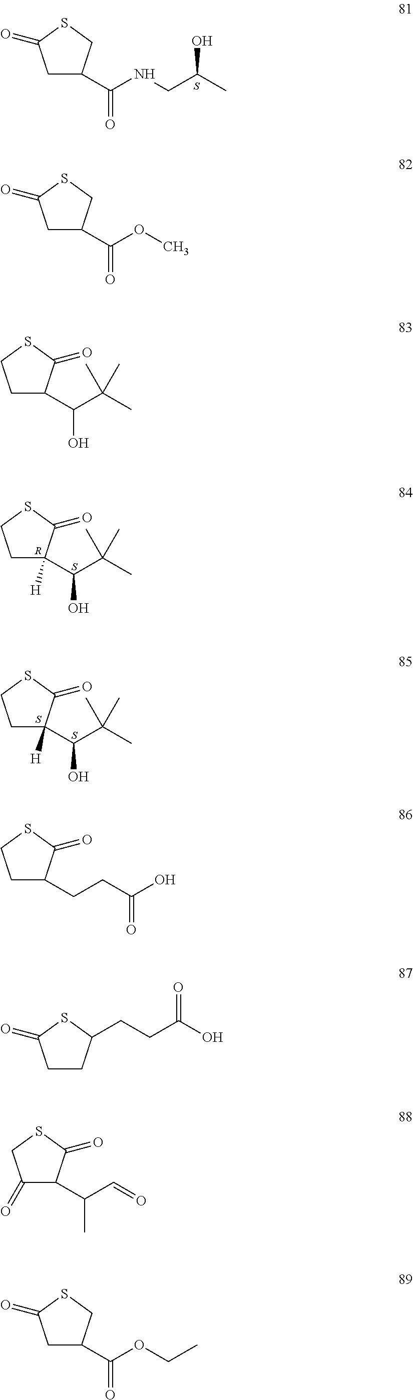 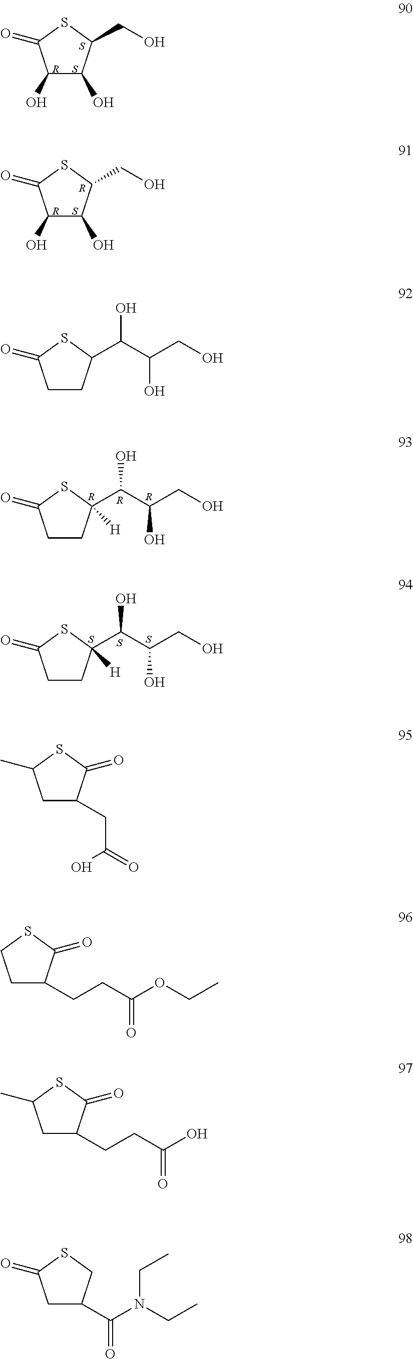 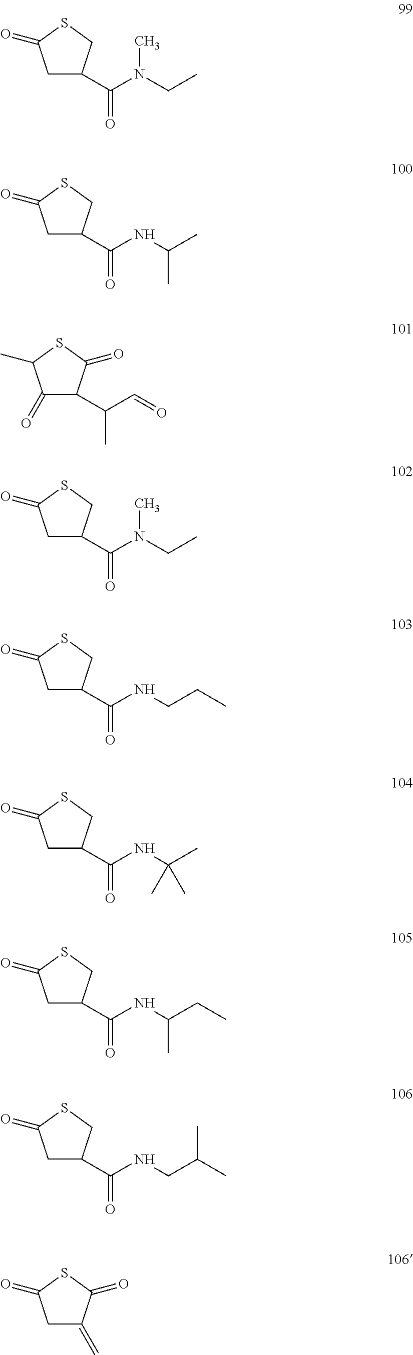 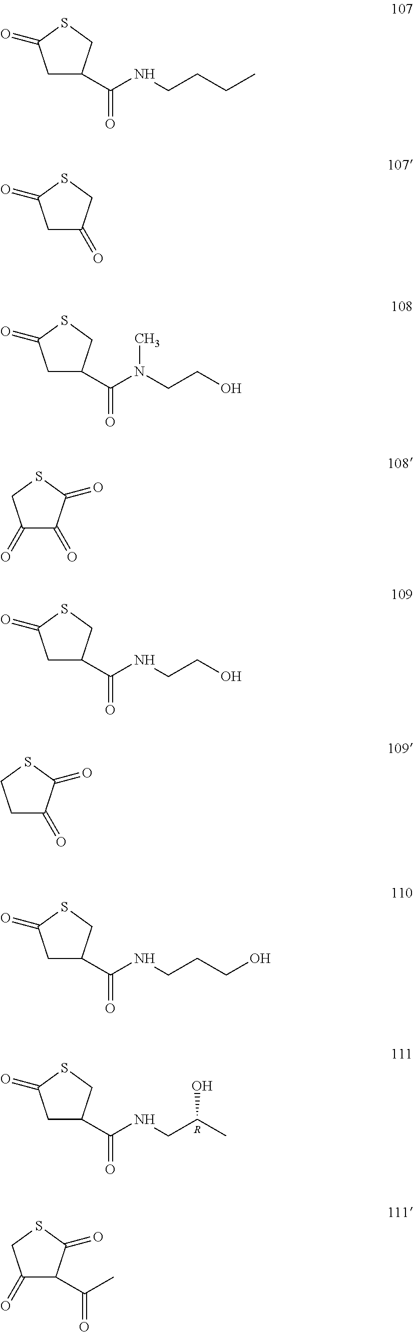 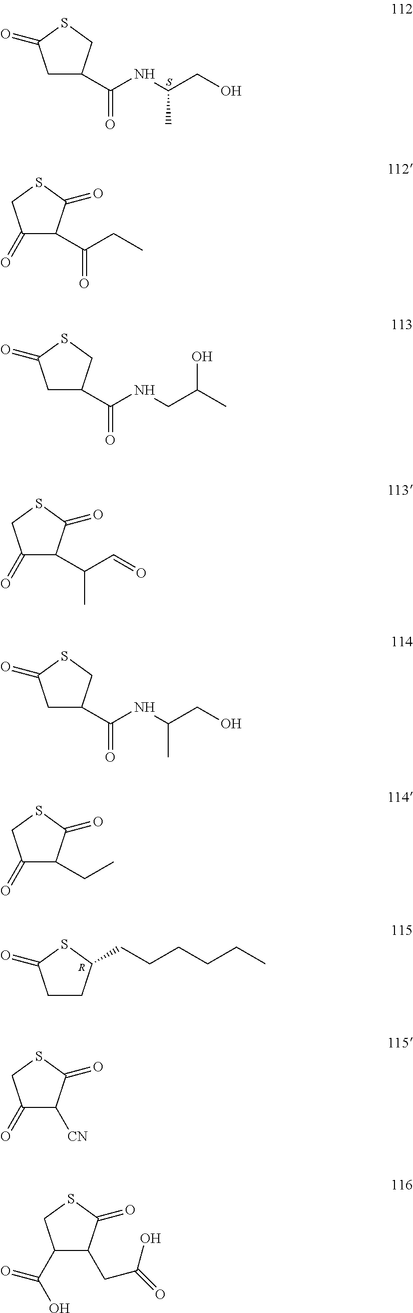 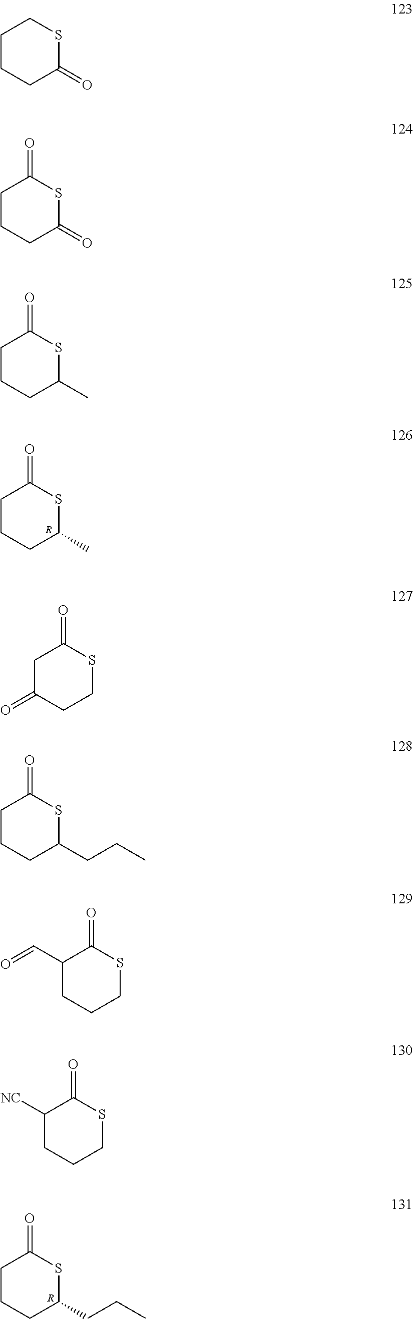 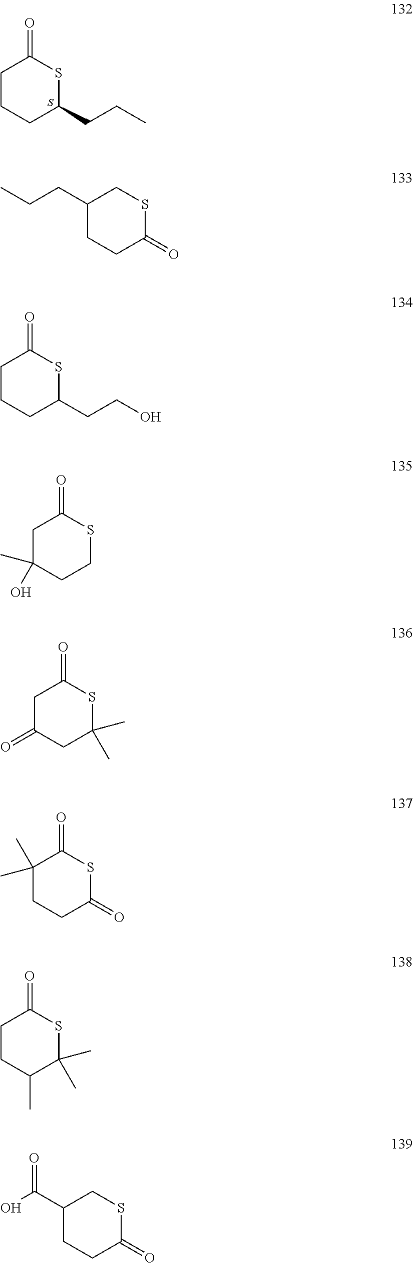 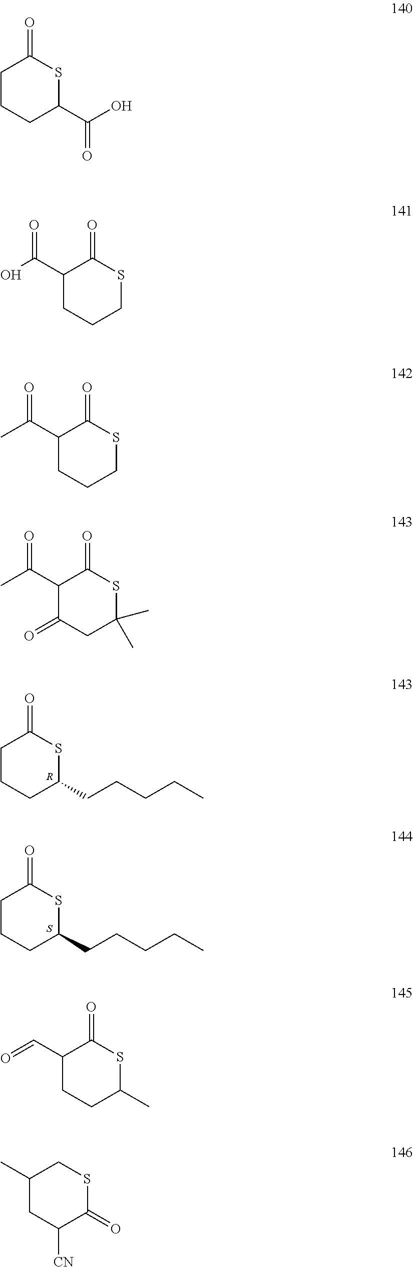 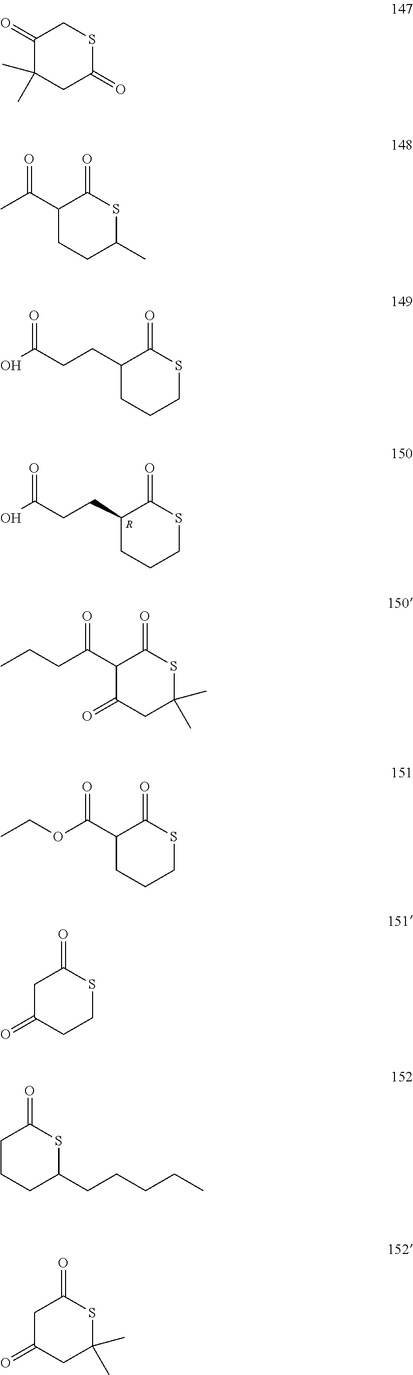 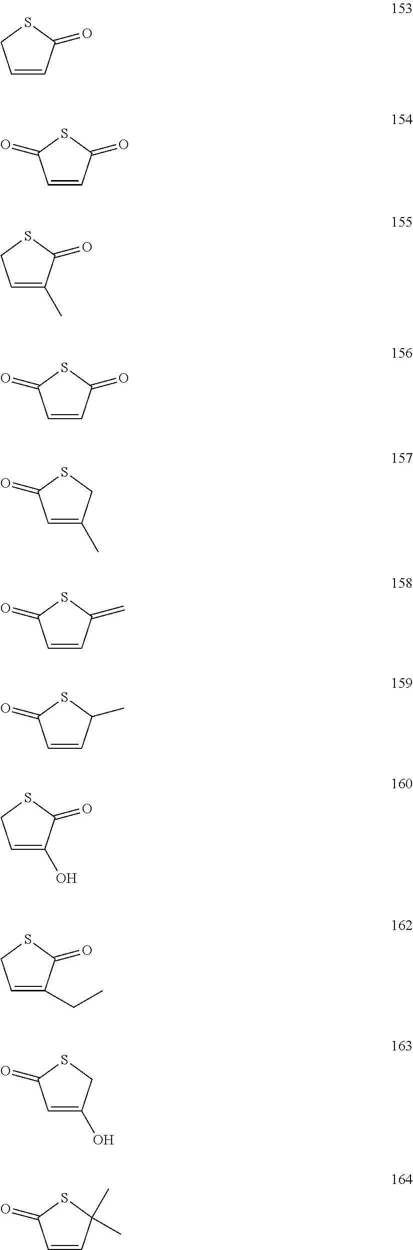 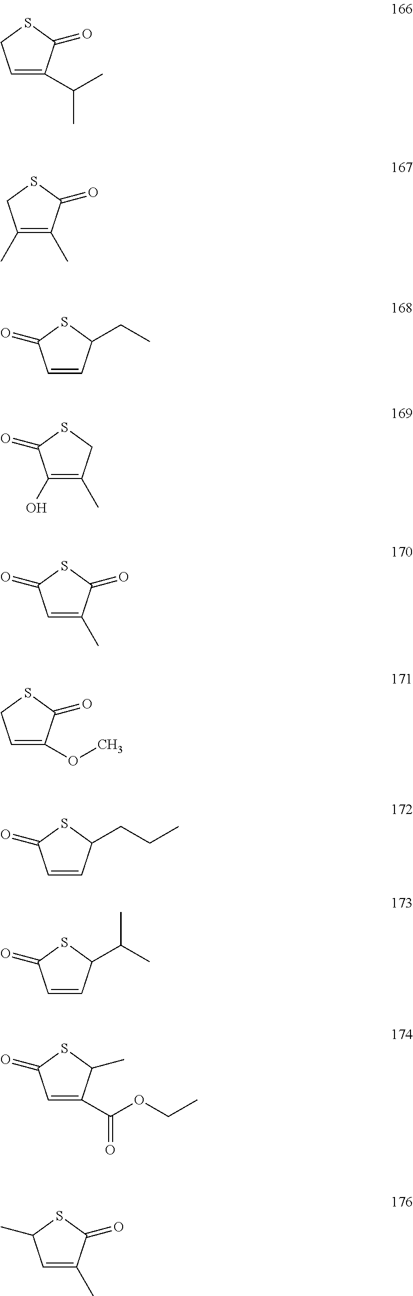 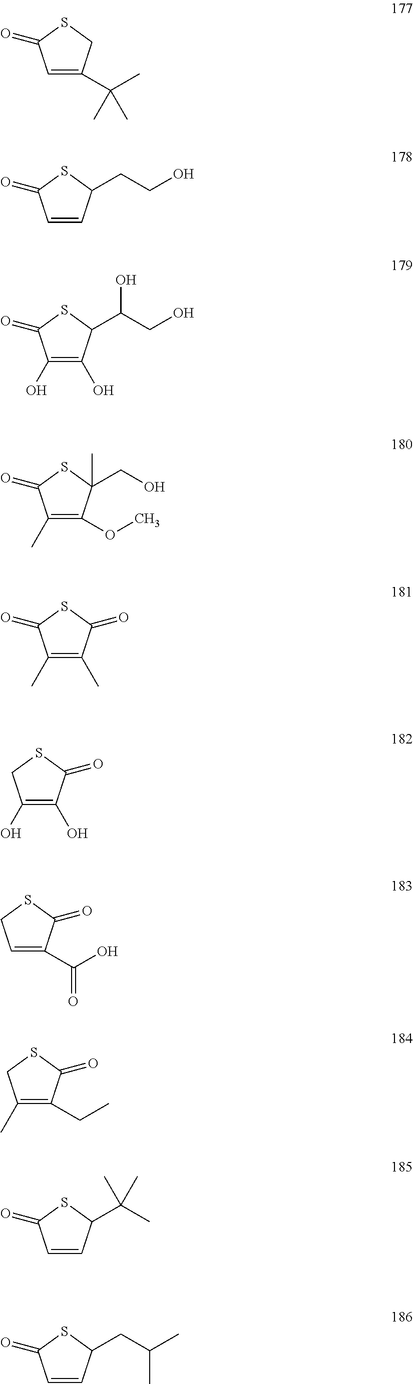 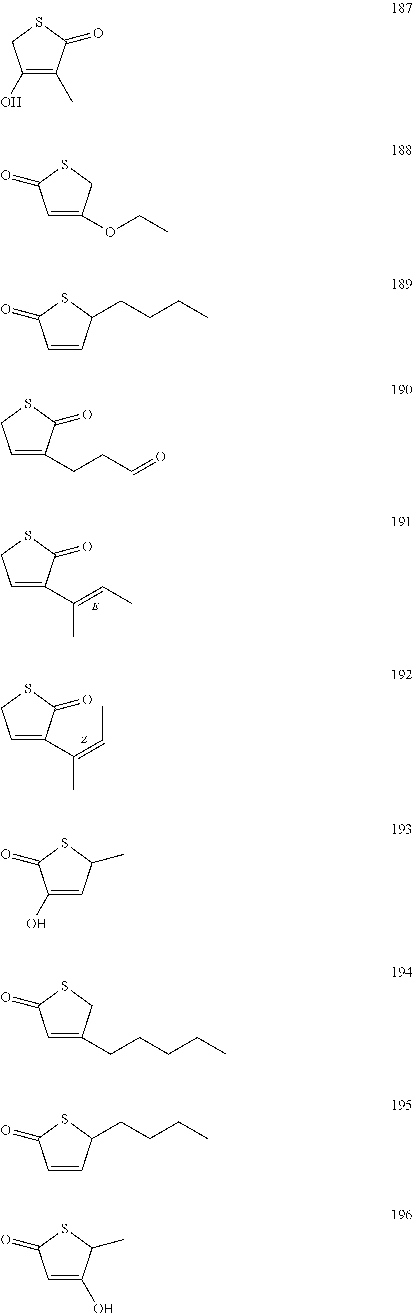 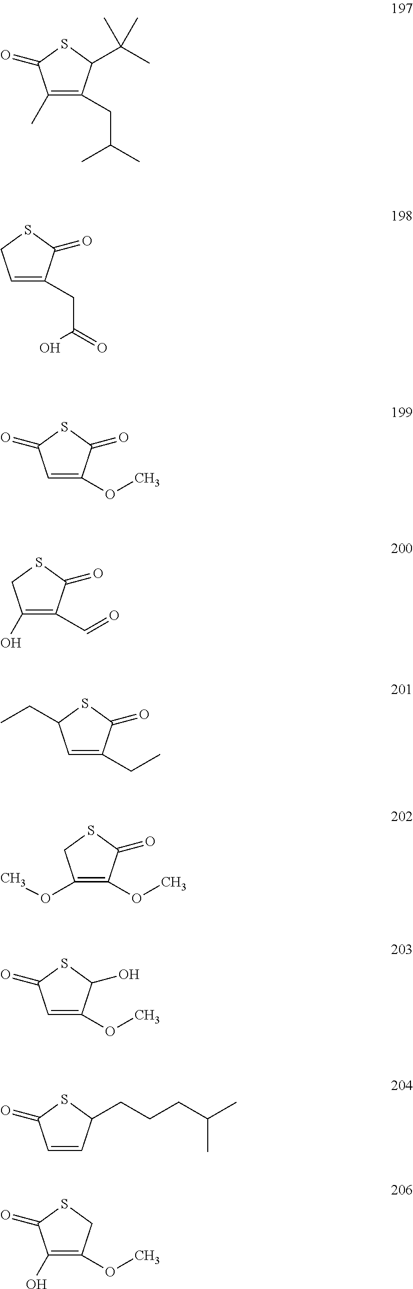 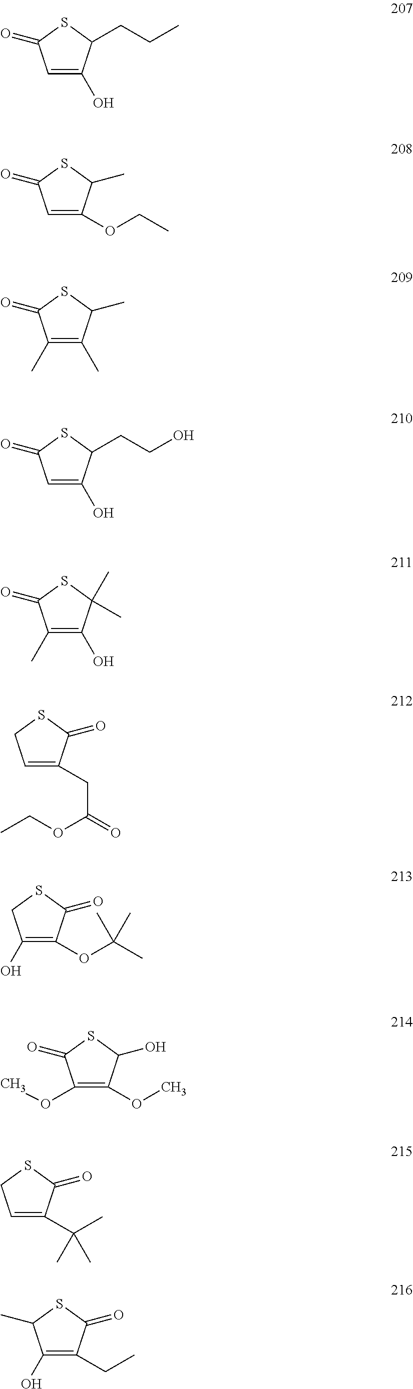 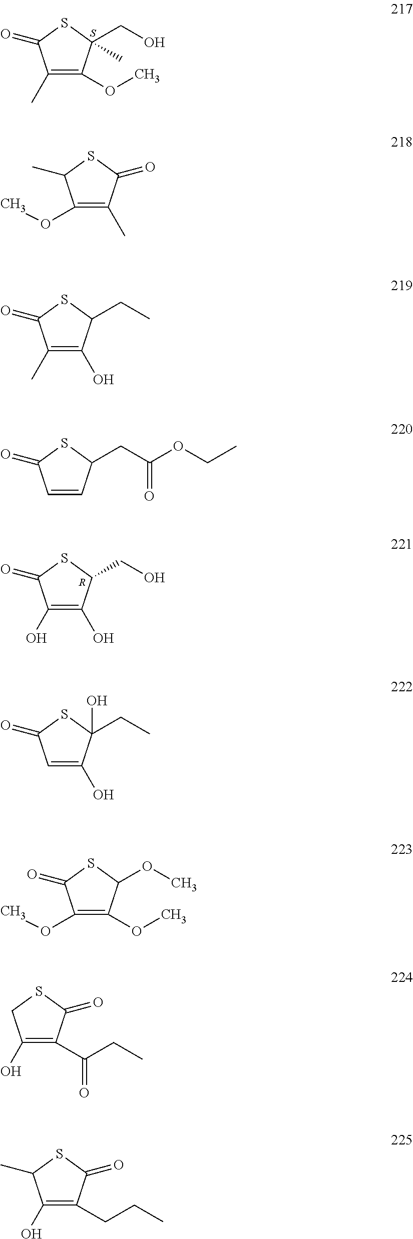 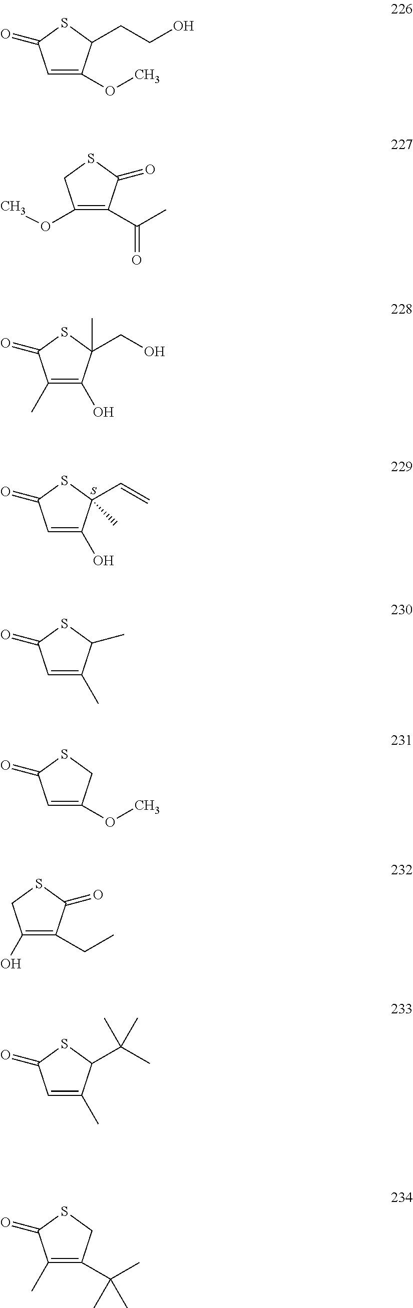 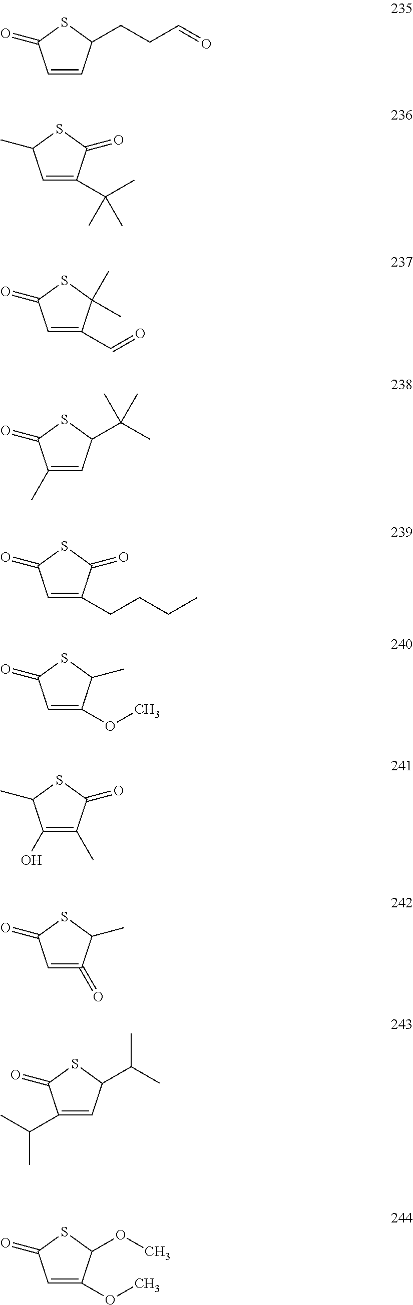 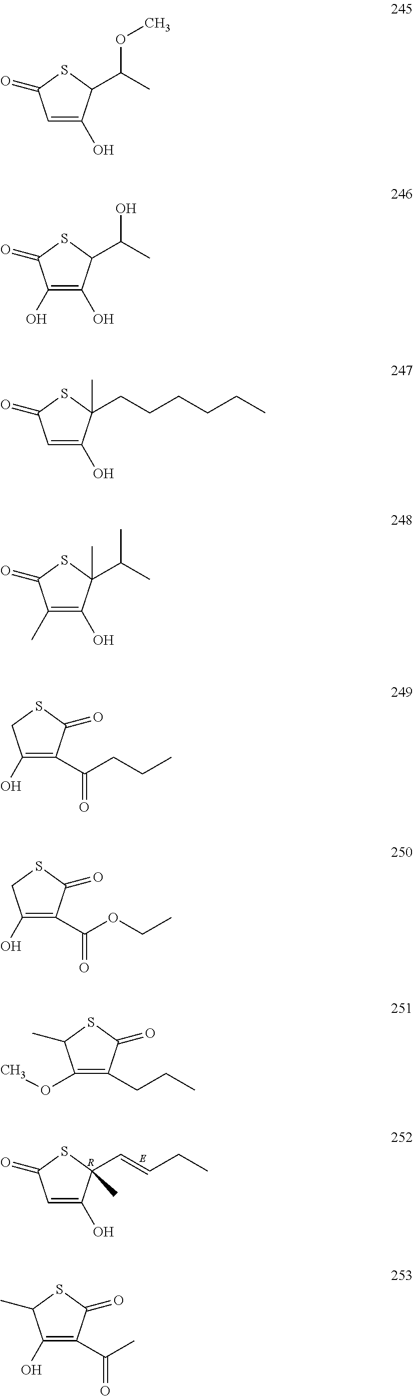 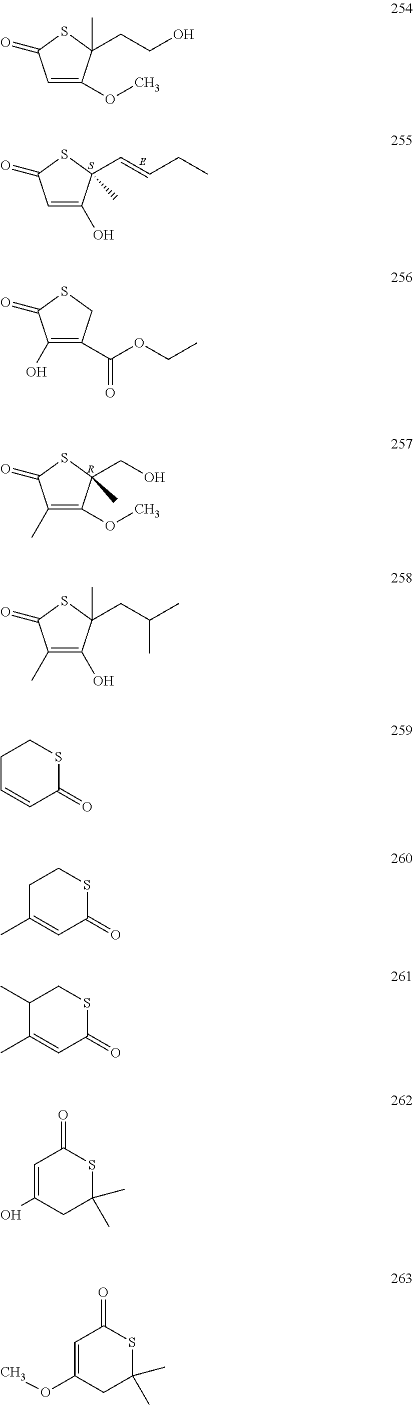 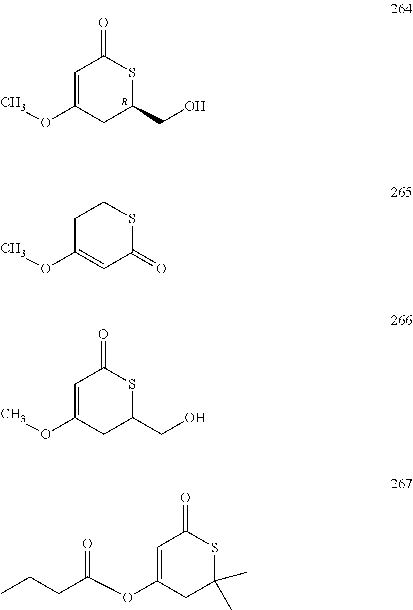
or organic or mineral acid or base salts thereof, optical isomers thereof, geometrical isomers thereof, tautomers thereof, or solvates thereof.

25. The composition according to claim 24, wherein the at least one compound of formula (I) is chosen from the compounds of formulae (38) to (122) and (153) to (258).

26. The composition according to claim 18, wherein the at least one compound of formula (I) is present in an amount ranging from 0.01% to 50% by weight, relative to the total weight of the composition.

27. The composition according to claim 18, wherein the composition comprises one or more functionalized silicone comprising at least one functional group chosen from amino groups, alkoxy groups, or hydroxyl groups.

28. The composition according to claim 18, wherein the composition comprises at least one amino silicone chosen from:
a) the polysiloxanes according to formula (A): 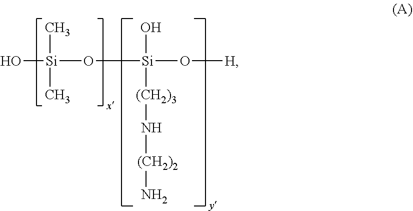
wherein x′ and y′ are integers such that the weight-average molecular weight (Mw) is between 5000 and 500 000 approximately;
b) the amino silicones corresponding to formula (B):
R′ₐG₃₋ₐ-Si(OSiG₂)ₙ-(OSiG_{b}R′_{2-b})ₘ—O-SiG₃₋ₐ-R′ₐ (B)
wherein:
G, which may be identical or different, denotes a hydrogen atom, a phenyl, OH, C₁-C₆ alkyl or C₁-C₆ alkoxy group,
a, which may be identical or different, denotes 0 or an integer from 1 to 3,
b denotes 0 or 1,
m and n are numbers such that the sum (n+m) ranges from 1 to 2000, n optionally denoting a number from 0 to 1999, and m optionally denoting a number from 1 to 2000;
R′, which may be identical or different, denotes a monovalent radical of formula —C_{q}H_{2q}L, wherein q is a number ranging from 2 to 8, and L is an optionally quaternized amino group chosen from the following groups:
—N(R″)₂; —N+(R″)₃ A-; —NR″-Q-N(R″)₂, or —NR″-Q-N+(R″)₃ A-,
wherein R″, which may be identical or different, is chosen from hydrogen, phenyl, benzyl, or a saturated monovalent hydrocarbon-based radical; Q is a linear or branched group of formula C₁H₂₁, wherein r is an integer ranging from 2 to 6; and A- is an anion;
c) the amino silicones corresponding to formula (H): 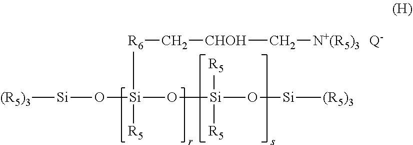
wherein:
R₅ represents a monovalent hydrocarbon-based radical containing from 1 to 18 carbon atoms;
R₆ represents a divalent hydrocarbon-based radical;
Q⁻ is an anion chosen from a halide ion or an organic acid salt;
r represents a mean statistical value ranging from 2 to 20;
s represents a mean statistical value ranging from 20 to 200;
d) the quaternary ammonium silicones of formula (I): 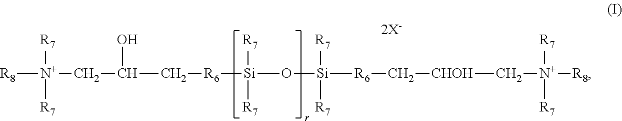
wherein:
R7, which may be identical or different, is chosen from a monovalent hydrocarbon-based radical containing from 1 to 18 carbon atoms, or a C₂-C₁₈ alkenyl radical or a ring comprising 5 or 6 carbon atoms;
R₆ is chosen from a divalent hydrocarbon-based radical, or a divalent C₁-C₁₈ alkyleneoxy radical linked to the Si via an SiC bond;
R₈, which may be identical or different, is chosen from a hydrogen atom, a monovalent hydrocarbon-based radical containing from 1 to 18 carbon atoms, and, a C₂-C₁₈ alkenyl radical, or a radical —R₆—NHCOR₇;
X⁻ is an anion chosen from a halide ion or an organic acid salt;
r represents a mean statistical value ranging from 2 to 200;
e) the amino silicones of formula (J): 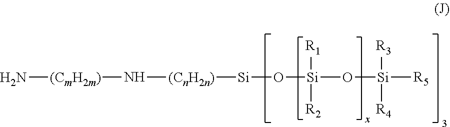
wherein:
R₁, R₂, R₃ and R₄, which may be identical or different, are chosen from a C₁-C₄ alkyl radical or a phenyl group,
R₅ is chosen from a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;
f) multiblock polyoxyalkylenated amino silicones, of the type (AB)n, A being a polysiloxane block and B being a polyoxyalkylene block comprising at least one amine group.

29. The composition according to claim 18, wherein the composition comprises one or more amino silicones chosen from:
the “trimethylsilyl amodimethicone” silicones corresponding to formula (C): 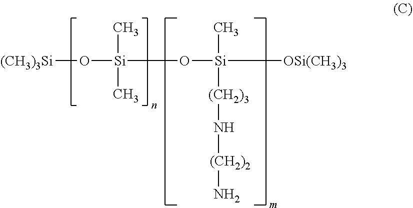
wherein m and n are numbers such that the sum (n+m) ranges from 1 to 2000, n optionally denotes a number from 0 to 1999, and for m optionally denotes a number from 1 to 2000;
the silicones of formula (D) below: 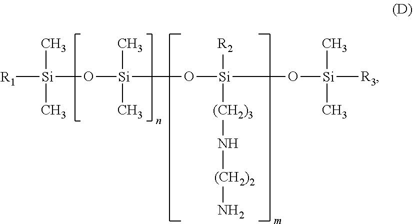
wherein:
m and n are numbers such that the sum (n+m) ranges from 1 to 1000; n optionally denotes a number from 0 to 999, and m optionally denotes a number from 1 to 1000;
R1, R2, and R3, which may be identical or different, are chosen from a hydroxyl or C1-C4 alkoxy radical, wherein at least one of the radicals R1 to R3 is an alkoxy radical;
the silicones of formula (E) below: 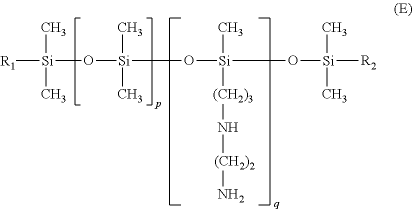
wherein:
p and q are numbers such that the sum (p+q) ranges from 1 to 1000; p optionally denotes a number from 0 to 999, and q optionally denotes a number from 1 to 1000, in particular from 1 to 10 and more particularly from 1 to 5;
R1 and R2, which are different, are chosen from a hydroxyl or C1-C4 alkoxy radical, wherein at least one of the radicals R1 or R2 is an alkoxy radical;
the silicones of formula (F) below: 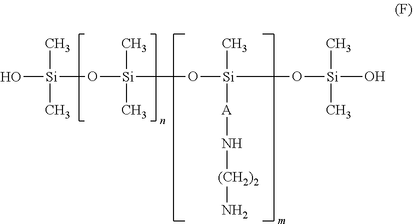
wherein:
m and n are numbers such that the sum (n+m) ranges from 1 to 2000, n is optionally a number ranging from 0 to 1999, and m is optionally a number ranging from 1 to 2000;
A is a linear or branched alkylene radical containing from 4 to 8 carbon atoms;
the silicones of formula (G) below: 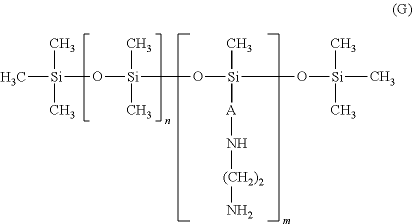
wherein:
m and n are numbers wherein the sum (n+m) ranges from 1 to 2000, n is optionally a number ranging from 0 to 1999, and m is optionally a number ranging from 1 to 2000;
A is a linear or branched alkylene radical containing from 4 to 8 carbon atoms.

30. The composition according to claim 18, wherein the at least one silicone is present in an amount ranging from 0.01% and 10%, inclusively, by weight relative to the total weight of the composition.

31. The composition according to claim 18, wherein the composition has a pH of less than or equal to 7.

32. The composition according to claim 18, wherein the composition does not comprise any thiol-based reducing agents.

33. A process for treating keratin materials, comprising applying to the keratin materials, of a composition comprising:
(a) at least one compound chosen from compounds of formula (I) below: 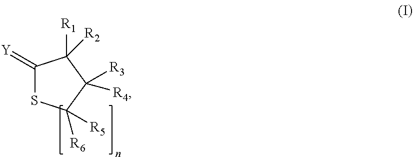
organic or mineral acid or base salts thereof, optical isomers thereof, geometrical isomers thereof, tautomers thereof, or solvates thereof;
wherein:
Y is chosen from an oxygen atom, a sulfur atom, or a group NR, wherein R is a hydrogen atom, a (C₁-C₆)alkyl group, or a hydroxy(C₁-C₆)alkyl group;
R₁, R₂, R₃, R₄, R₅ and R₆, which may be identical or different, are chosen from:
i) a hydrogen atom,
ii) hydroxyl,
iii) amino —NR′R″, wherein R′ and R″, which may be identical or different, are chosen from a hydrogen atom, a (C₁-C₆)alkyl, or a hydroxy(C₁-C₆)alkyl group;
iv) cyano,
v) —(Y′)ₚ—C(Y″)—(Y′″)_{q}—R₇, wherein R₇ is a hydrogen atom or a (C₁-C₆)alkyl group, p and q, which may be identical or different, are equal to 0 or 1, Y′, Y″ and Y′″, which may be identical or different, are chosen from an oxygen atom, sulfur atom, or NR, wherein R is a hydrogen atom, a (C₁-C₆)alkyl group, or a hydroxy(C₁-C₆)alkyl group,
vi) optionally substituted (C₁-C₆)alkyl,
vii) optionally substituted (C₂-C₆)alkenyl,
viii) optionally substituted (C₁-C₆)alkoxy,
or alternatively:
ix) R₁ and R₃ together form a covalent bond;
x) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl optionally substituted with one or more hydroxyl groups, and d) (C₂-C₆)alkenyl, e) —(Y′)ₚ—C(Y″)—C(Y′″)_{q}—R₇;
n represents an integer between 0 and 6 inclusive;
wherein:
when n is greater than or equal to 2, then the contiguous groups C(R₅)—R₆ are optionally identical or different;
and
(b) at least one silicone.

34. The process for treating the keratin materials according to claim 33, wherein applying the composition to the hair is followed by shaping the keratin materials by means of a heat treatment at a temperature of at least 100° C.

35. The process according to claim 34, wherein the shaping comprises straightening the keratin materials at a temperature between 100° C. and 300° C.
